SEQUENCE LISTING (16 sequences)

SEQ ID NO: 1 - DNA, 8136 bp, Artificial sequence
  Synthetic construct
  misc_feature: CHIKV S27 based vector; derived from Genbank # AF369024; containing native S27 5′ and 3′ UTRs and a SpeI cloning site
atggctgcgtgagacacacgtagcctaccagtttcttactgctctactctgcaaagcaagagattaagaacccatcatggatcctgtgtacgtggacatagacgctgacagcgcctttttgaaggccctgcaacgtgcgtaccccatgtttgaggtggaacctaggcaggtcacaccgaatgaccatgctaatgctagagcgttctcgcatctagctataaaactaatagagcaggaaattgatcccgactcaaccatcctggatattggtagtgcgccagcaaggaggatgatgtcggacaggaagtaccactgcgtttgcccgatgcgcagtgcagaagatcccgagagactcgccaattatgcgagaaagctagcatctgccgcaggaaaagtcctggacagaaacatctctggaaagatcggggacttacaagcagtaatggccgtgccagacacggagacgccaacattctgcttacacacagatgtatcatgtagacagagagcagacgtcgcgatataccaagacgtctatgctgtacacgcacccacgtcgctataccaccaggcgattaaaggggtccgattggcgtactgggtagggtttgacacaaccccgttcatgtacaatgccatggcgggtgcctacccctcatactcgacaaattgggcagatgagcaggtactgaaggctaagaacataggattatgttcaacagacctgacggaaggtagacgaggcaaattgtctattatgagaggaaaaaagctagaaccgtgcgaccgtgtgctgttctcagtagggtcaacgctctacccggaaagccgtaagctacttaagagctggcacctaccatcggtgttccatttaaagggcaagctcagcttcacatgccgctgtgatacagtggtttcgtgcgaaggctacgtcgttaagagaataacgatgagcccaggcctttacggaaaaaccacagggtatgcggtaacccaccacgcagacggattcctgatgtgcaagaccaccgacacggttgacggcgaaagagtgtcattctcggtgtgcacgtacgtgccggcgaccatttgtgatcaaatgaccggcatccttgctacagaagtcacgccggaggatgcacagaagctgttggtggggctgaaccagagaatagtggttaacggcagaacgcaacggaatacgaacaccatgaaaaactatatgattcccgtggtcgcccaagccttcagtaagtgggcaaaggagtgccggaaagacatggaagatgaaaaactcctgggggtcagagaaagaacactgacctgctgctgtctatgggcatttaagaagcagaaaacacacacggtctacaagaggcctgatacccagtcaattcagaaggttcaggccgagtttgacagctttgtggtaccgagcctgtggtcgtccgggttgtcaatcccgttgaggactagaatcaaatggttgttaagcaaggtgccaaaaaccgacctgaccccatacagcggggacgcccaagaagcccgggacgcagaaaaagaagcagaggaagaacgagaagcagaactgactcttgaagccctaccaccccttcaggcagcacaggaagatgttcaggtcgaaatcgacgtggaacagcttgaggacagagcgggtgcaggaataatagagactccgagaggagctatcaaagttactgcccaaccaacagaccacgtcgtgggagagtacttggttctttccccgcagaccgtactacgtagccaaaagcttagcctgattcacgctttggcggagcaagtgaagacgtgcacgcacagcggacgagcagggaggtatgcggtcgaagcgtacgacggcagagtcctagtgccctcaggctacgcaatctcgcctgaagacttccagagcctaagcgaaagcgcaacgatggtgtacaacgaaagagagttcgtaaacagaaagctacaccatattgcgatgcatggaccagccctgaacaccgacgaagagtcgtatgagctggtgagggcagagaggacagaacacgagtacgtctacgacgtggaccagagaagatgctgtaagaaggaagaagctgcaggactggtactggtgggcgacttgactaatccgccctaccacgaattcgcatatgaagggctaaaaatccgccctgcctgcccatacaaaattgcagtcataggagtcttcggagtaccaggatctggcaagtcagctattatcaagaacctagttaccaggcaagacctggtgactagcggaaagaaagaaaactgccaagaaatcaccaccgacgtgatgagacagagaggtctagagatatctgcacgtacggttgactcgctgctcttgaatggatgtaacagaccagtcgacgtgttgtacgtagacgaggcgtttgcgtgccactctggaacgttacttgcattgatcgccttggtgagaccaagacagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgactgccattgtgtcatcgttgcattacgaaggcaaaatgcgcactacgaatgagtacaacaagccgattgtagtggacactacaggctcaacaaaacctgaccctggagatctcgtgttaacgtgcttcagaggatgggttaaacaactgcaaattgactatcgtggacacgaggtcatgacagcagccgcatcccaagggttaaccagaaaaggagtttacgcagttaggcaaaaagttaacgaaaacccgctttatgcatcaacgtcagagcacgtcaacgtactcctaacgcgtacggaaggtaaactggtatggaagacactctccggtgacccgtggataaagacgctgcagaacccaccgaaaggaaacttcaaagcaactattaaggagtgggaggtggagcatgcatcaataatggcgggcatctgcagtcaccaaatgacctttgatacattccaaaacaaagccaacgtttgttgggctaagagtttggtccctatcctcgaaacagcggggataaaactaaacgacaggcagtggtcccagataattcaagccttcaaagaagacaaagcatattcacccgaagtagccctgaatgaaatatgcacgcgcatgtatggggtggatctagacagcgggctattttctaaaccgttggtgtctgtgtattacgcggataaccactgggataataggcctggagggaagatgttcggattcaaccccgaggcagcatccattctagaaagaaagtatccatttacaaaagggaagtggaacatcaacaagcagatctgcgtgactaccaggaggatagaagacttcaaccctaccaccaacattataccggccaacaggagactaccacactcattagtggccgaacaccgcccagtaaaaggggaaagaatggaatggctggttaacaagataaacggccaccacgtgctcctggtcagtggctgtagccttgcactgcctactaagagagtcacttgggtagcgccactaggtgtccgcggagcggactatacatacaacctagagttgggtctgccagcaacgcttggtaggtatgacctagtggtcataaacatccacacaccttttcgcatacaccattatcaacagtgcgtagaccacgcaatgaaactgcaaatgctcgggggtgactcattgagactgctcaaaccgggtggctctctattgatcagagcatatggttacgcagatagaaccagtgaacgagtcatctgcgtattgggacgcaagtttagatcatctagagcgttgaaaccaccatgtgtcaccagcaacactgagatgttttttctattcagcaactttgacaatggcagaaggaatttcacaactcatgtcatgaacaatcaactgaatgcagcctttgtaggacaggccacccgagcaggatgtgcaccgtcgtaccgggtaaaacgcatggatatcgcgaagaacgatgaagagtgcgtagtcaacgccgccaaccctcgcgggttaccaggtgacggtgtttgcaaggcagtatacaaaaaatggccggagtcctttaagaacagtgcaacaccagtgggaaccgcaaaaacagtcatgtgcggtacgtatccagtaatccacgccgttggaccaaacttctctaattattcggagtctgaaggggaccgagaattggcggctgcctatcgagaagtcgcaaaggaggtaactagactgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtggagctgctggatgagcacatctccatagactgcgatgttgttcgcgtgcaccctgacagcagcttggcaggcagaaaaggatacagcaccacggaaggcgcactgtactcatatctagaagggacccgttttcaccaaacggcagtggatatggcagagatatatactatgtggccaaagcaaacagaggccaacgagcaagtttgcctatatgccctgggggaaagtattgaatcgatcaggcagaaatgcccggtggatgatgcagatgcatcatctcccccgaaaactgtcccgtgcctctgccgttacgccatgacaccagaacgcgttacccgacttcgcatgaaccatgtcacaagcataattgtgtgttcttcgtttccccttccaaagtacaaaatagaaggagtgcaaaaagtcaaatgctccaaggtaatgctatttgaccacaacgtgccatcgcgcgtaagtccaagggaatacagaccttcccaggagtctgtacaggaagcgagtacgaccacgtcactgacgcatagccaattcgatctaagcgttgacggcaagatactgcccgtcccgtcagacctggatgctgacgccccagccctagaaccagcccttgacgacggggcgatacacacgttgccatctgcaaccggaaaccttgcggccgtgtctgactgggtaatgagcaccgtacctgtcgcgccgcccagaagaaggcgagggagaaacctgactgtgacatgcgacgagagagaagggaatataacacccatggctagcgtccgattctttagggcagagctgtgtccagtcgtacaagaaacagcggagacgcgtgacacagctatgtctcttcaggcaccgccgagtaccgccacggaactgagtcacccgccgatctccttcggtgcaccaagcgagacgttccccatcacatttggggacttcaacgaaggagaaatcgaaagcttgtcttctgagctactaactttcggagacttcctacccggagaagtggatgatttgacagatagcgactggtccacgtgctcagacacggacgacgagttacgactagacagggcaggtgggtatatattctcgtcggacactggtccaggtcatttacaacagaagtcagtacgccagtcagtgctgccggtgaacaccctggaggaagtccacgaggagaagtgttacccacctaagctggatgaagcaaaggagcaactactacttaagaaactccaggagagtgcatccatggccaacagaagcaggtatcagtcgcgcaaagtagaaaacatgaaagcaacaatcatccagagactaaagagaggctgtagattatacttaatgtcagagaccccaaaagtccctacctaccggaccacatatccggcgcctgtgtactcgcctccgattaacgtccgactgtccaaccccgagtccgcagtggcagcatgcaatgagttcttggctagaaactatccaactgtttcatcataccaaatcaccgacgagtatgatgcatatctagacatggtggacgggtcggagagttgtctggaccgagcgacattcaatccgtcaaaacttaggagctacccaaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaaattcgcatgcaaccaagaatactgggaagaatttgctgccagccctatcaggataacaactgagaatttaacaacctatgttactaaactaaaggggccaaaagcagcagcgctatttgcaaaaacccataatctgctgccactgcaggaagtgccaatggataggttcacagtagacatgaaaagggatgtgaaggtgactcctggtacaaagcacacagaggaaagacctaaggtacaggttatacaggcggctgaacccttggcaacagcatacctatgtgggattcacagagagctggttaggaggctgaacgccgtcctcctacccaatgtacatacactatttgacatgtctgccgaggatttcgatgccatcatagccgcacactttaagccaggagacactgttttagaaacggacatagcctcctttgataagagccaagatgattcacttgcgcttactgctttaatgctgttagaggatttaggggtggatcactccctgttggacttgatagaggctgctttcggagagatttccagctgtcatctaccgacaggtacgcgcttcaagttcggcgccatgatgaaatctggtatgttcctaactctgttcgtcaacacactgctaaatatcaccatcgccagccgagtgctggaagatcgtctgacaaaatccgcgtgcgcagccttcatcggcgacgacaacataatacatggagtcgtctccgatgaattgatggcagccagatgcgccacttggatgaacatggaagtgaagatcatagatgcagttgtatcccagaaagccccttacttttgtggagggtttatactgcacgatatcgtgacaggaacagcttgcagagtggcagacccgctaaaaaggctatttaaactgggcaaaccgctagcggcaggtgacgaacaagatgaggatagaagacgagcgctggctgacgaagtggtcagatggcaacgaacagggctaattgatgagttggagaaagcggtatactctaggtatgaagtgcagggtatatcagttgtggtaatgtccatggccacctttgcaagctccagatccaacttcgagaagctcagaggacccgtcgtaactttgtacggcggtcctaaataggtacgcactacagctacctattttgcagaagccgacagtaagtacctaaacactaatcagctacaatagtcagcatagtacatttcatctgactaatactacaacaccaccaccactagtaacttgacgactaagcatgaaggtatatgtgtcccctaagagacacaccgtatatagctaataatctgtagatcaaagggctatataacccctgaatagtaacaaaatacaaaatcactaaaaattataaaaaaaaaaaaaaaaaaacagaaaaatatataaataggtatacgtgtcccctaagagacacattgtatgtaggtgataagtatagatcaaagggccgaacaacccctgaatagtaacaaaatataaaaattaataaaaatcataaaatagaaaaaccataaacagaagtagttcaaagggctataaaaacccctgaatagtaacaaaacataaaactaataaaaatcaaatgaataccataattggcaaacggaagagatgtaggtacttaagcttcctaaaagcagccgaactcactttgagatgtaggcatagcataccgaactcttccacgattctccgaacccacagggacgtaggagatgttattttgtttttaatatttc

SEQ ID NO: 2 - DNA, 8084 bp, Artificial sequence
  Synthetic construct
  misc_feature: CHIKV DRDE; derived from Genbank # EF210157; containing native DRDE; 5′ and 3′ UTRs and a SpeI cloning site
atggctgcgtgagacacacgtagcctaccagtttcttactgctctactctgcaaagcaagagattaataacccatcatggatcctgtgtacgtggacatagacgctgacagcgcctttttgaaggccctgcaacgtgcgtaccccatgtttgaggtggaaccaaggcaggtcacaccgaatgaccatgctaatgctagagcgttctcgcatctagctataaaactaatagagcaggaaattgaccccgactcaaccatcctggatatcggcagtgcgccagcaaggaggatgatgtcggacaggaagtaccactgcgtctgcccgatgcgcagtgcggaagatcccgagagactcgctaattatgcgagaaagctagcatctgccgcaggaaaagtcctggacagaaacatctctggaaagatcggggacttacaagcagtaatggccgtgccagacaaggagacgccaacattctgcttacacacagacgtctcatgtagacagagagcagacgtcgctatataccaagacgtctatgctgtacacgcacccacgtcgctataccaccaggcgattaaaggggtccgagtggcgtactgggttgggttcgacacaaccccgttcatgtacaatgccatggcgggtgcctacccctcatactcgacaaactgggcagatgagcaggtactgaaggctaagaacataggattatgttcaacagacctgacggaaggtagacgaggcaagttgtctattatgagagggaaaaagctaaaaccgtgcgaccgtgtgctgttctcagtagggtcaacgctctacccggaaagccgcaagctacttaagagctggcacctgccatcggtgttccatttaaagggcaaactcagcttcacatgccgctgtgatacagtggtttcgtgtgagggctacgtcgttaagagaataacgatgagcccaggcctttatggaaaaaccacagggtatgcggtaacccaccacgcagacggattcctgctgtgcaagactaccgacacggttgacggcgaaagagtgtcattctcggtgtgcacatacgtgccggcgaccatttgtgatcaaatgaccggcatccttgctacagaagtcacgccggaggatgcacagaagctgttggtggggctgaaccagagaatagtggttaacggcagaacgcaacggaatatgaacaccatgaaaaattatctgcttcccgtggtcgcccaagccttcagtaagtgggcaaaggagtgccggaaagacatggaagatgaaaaactcctgggggtcagagaaagaacactgacctgctgctgtctatgggcattcaagaagcagaaaacacacacggtctacaagaggcctgatacccagtcaattcagaaggttcaggccgagtttgacagctttgtggtaccgagtctgtggtcgtccgggttgtcaatccctttgaggactagaatcaaatggttgttaagcaaggtgccaaaaaccgacctgatcccatacagcggagacgcccgagaagcccgggacgcagaaaaagaagcagaggaagaacgagaagcagaactgactcgcgaagccctaccacctctacaggcagcacaggaagatgttcaggtcgaaatcgacgtggaacagcttgaggacagagcgggcgcaggaataatagagactccgagaggagctatcaaagttactgcccaaccaacagaccacgtcgtgggagagtacctggtactctccccgcagaccgtactacgtagccagaagctcagtctgattcacgctttggcggagcaagtgaagacgtgcacgcacaacggacgagcagggaggtatgcggtcgaagcgtacgacggccgagtcctagtgccctcaggctatgcaatctcgcctgaagacttccagagtctaagcgaaagcgcgacgatggtgtataacgaaagagagttcgtaaacagaaagctacaccatattgcgatgcacggaccagccctgaacaccgacgaagagtcgtatgagctggtgagggcagagaggacagaacacgagtacgtctacgacgtggatcagagaagatgctgtaagaaggaagaagccgcaggactggtactggtgggcgacttgactaatccgccctaccacgaattcgcatatgaagggctaaaaatccgccctgcctgcccatacaaaattgcagtcataggagtcttcggagtaccgggatctggcaagtcagctattatcaagaacctagttaccaggcaggacctggtgactagcggaaagaaagaaaactgccaagaaatcaccaccgacgtgatgagacagagaggtctagagatatctgcacgtacggttgactcgctgctcttgaatggatgcaacagaccagtcgacgtgttgtacgtagacgaggcgtttgcgtgccactctggaacgctacttgctttgatcgccttggtgagaccaaggcagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgaccgccattgtgtcatcgttgcattacgaaggcaaaatgcgcactacgaatgagtacaacaagccgatcgtagtggacactacaggctcaacaaaacctgaccctggagacctcgtgttaacgtgcttcagagggtgggttaaacaactgcaaattgactatcgtggatacgaggtcatgacagcagccgcatcccaagggttaaccagaaaaggagtttacgcagttagacaaaaagttaatgaaaacccgctctatgcatcaacgtcagagcacgtcaacgtactcctaacgcgtacggaaggtaaactggtatggaagacactttccggcgacccgtggataaagacgctgcagaacccaccgaaaggaaacttcaaagcaactattaaggagtgggaggtggagcatgcatcaataatggcgggcatctgcagtcaccaaatgaccttcgatacattccaaaataaagccaacgtttgttgggctaagagcttggtccctatcctcgaaacagcggggataaaactaaatgataggcagtggtctcagataattcaagccttcaaagaagacaaagcatactcacctgaagtagccctgaatgaaatatgtacgcgcatgtatggggtggatctagacagcgggctattttctaaaccgttggtgtctgtgtattacgcggataaccactgggataataggcctggagggaaaatgttcggatttaaccccgaggcagcatccattctagaaagaaagtatccattcacaaaagggaagtggaacatcaacaagcagatctgcgtgactaccaggaggatagaagactttaaccctaccaccaacatcataccggccaacaggagactaccacactcattagtggccgaacaccgcccagtaaaaggggaaagaatggaatggctggttaacaagataaacggccaccacgtgctcctggtcagtggctataaccttgcactgcctactaagagagtcacttgggtagcgccgttaggtgtccgcggagcggactacacatacaacctagagttgggtctgccagcaacgcttggtaggtatgaccttgtggtcataaacatccacacaccttttcgcatacaccattaccaacagtgcgtcgaccacgcaatgaaactgcaaatgctcgggggtgactcattgagactgctcaaaccgggcggctctctattgatcagagcatatggttacgcagatagaaccagtgaacgagtcatctgcgtattgggacgcaagtttagatcgtctagagcgttgaaaccaccatgtgtcaccagcaacactgagatgtttttcctattcagcaactttgacaatggcagaaggaatttcacaactcatgtcatgaacaatcaactgaatgcagccttcgtaggacaggtcacccgagcaggatgtgcaccgtcgtaccgggtaaaacgcatggacatcgcgaagaacgatgaagagtgcgtagtcaacgccgctaaccctcgcgggttaccgggtgacggtgtttgcaaggcagtatacaaaaaatggccggagtcctttaagaacagtgcaacaccagtgggaaccgcaaaaacagttatgtgcggtacgtatccagtaatccacgctgttggaccaaacttctctaattattcggagtctgaaggggaccgggaattggcagctgcctatcgagaagtcgcaaaggaagtaactaggctgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtagagctgctggatgagcacatctccatagactgcgatattgttcgcgtgcaccctgacagcagcttggcaggcagaaaaggatacagcaccacggaaggcgcactgtactcatatctagaagggacccgttttcatcagacggctgtggatatggcggagatacatactatgtggccaaagcaaacagaggccaatgagcaagtctgcctatatgccctgggggaaagtattgaatcgatcaggcagaaatgcccggtggatgatgcagacgcatcatctccccccaaaactgtcccgtgcctttgccgttacgctatgactccagaacgcgtcacccggcttcgcatgaaccacgtcacaagcataattgtgtgttcttcgtttcccctcccaaagtacaaaatagaaggagtgcaaaaagtcaaatgctctaaggtaatgctatttgaccacaacgtgccatcgcgcgtaagtccaagggaatatagatcttcccaggagtctgcacaggaggcgagtacaatcacgtcactgacgcatagtcaattcgacctaagcgttgatggcgagatactgcccgtcccgtcagacctggatgctgacgccccagccctagaaccagcactagacgacggggcgacacacacgctgccatccacaaccggaaaccttgcggccgtgtctgactgggtaatgagcaccgtacctgtcgcgccgcccagaagaaggcgagggagaaacctgactgtgacatgtgacgagagagaagggaatataacacccatggctagcgtccgattctttagggcagagctgtgtccggtcgtacaagaaacagcggagacgcgtgacacagcaatgtctcttcaggcaccaccgagtaccgccacggaaccgaatcatccgccgatctccttcggagcatcaagcgagacgttccccattacatttggggacttcaacgaaggagaaatcgaaagcttgtcttctgagctactaactttcggagacttcttaccaggagaagtggatgacttgacagacagcgactggtccacgtgctcagacacggacgacgagttatgactagacagggcaggtgggtatatattctcgtcggacaccggtccaggtcatttacaacagaagtcagtacgccagtcagtgctgccggtgaacaccctggaggaagtccacgaggagaagtgttacccacctaagctggatgaagcaaaggagcaactattacttaagaaactccaggagagtgcatccatggccaacagaagcaggtatcagtcgcgcaaagtagaaaacatgaaagcagcaatcatccagagactaaagagaggctgtagactatacttaatgtcagagaccccaaaagtccctacttaccggactacatatccggcgcctgtgtactcgcctccgatcaacgtccgattgtccaatcccgagtccgcagtggcagcatgcaatgagttcttagctagaaactatccaactgtctcatcataccaaattaccgacgagtatgatgcatatctagacatggtggacgggtcggagagttgcctggaccgagcgacattcaatccgtcaaaactcaggagctacccgaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaagttcgcatgcaaccaagaatactgggaagaatttgctgccagccctattaggataacaactgagaatttagcaacctatgttactaaactaaaagggccaaaagcagcagcgctattcgcaaaaacccataatctactgccactacaggaagtaccaatggataggttcacagtagatatgaaaagggacgtgaaggtgactcctggtacaaagcatacagaggaaagacctaaggtgcaggttatacaggcggctgaacccttggcgacagcatacctatgtgggattcacagagagctggttaggaggctgaacgccgtcctcctacccaatgtacatacactatttgacatgtctgccgaggatttcgatgccatcatagccgcacactttaagccaggagacactgttttggaaacggacatagcctcctttgataagagccaagatgattcacttgcgcttactgctttgatgctgttagaggatttaggggtggatcactccctgctggacttgatagaggctgctttcggagagatttccagctgtcacctaccgacaggtacgcgcttcaagttcggcgccatgatgaaatcaggtatgttcctaactctgttcgtcaacacattgttaaacatcaccatcgccagccgagtgctggaagatcgtctgacaaaatccgcgtgcgcggccttcatcggcgacgacaacataatacatggagtcgtctccgatgaattgatggcagccagatgtgccacttggatgaacatggaagtgaagatcatagatgcagttgtatccttgaaagccccttacttttgtggagggtttatactgcacgatactgtgacaggaacagcttgcagagtggcagacccgctaaaaaggctttttaaactgggcaaaccgctagcggcaggtgacgaacaagatgaagatagaagacgagcgctggctgacgaagtgatcagatggcaacgaacagggctaattgatgagctggagaaagcggtatactctaggtacgaagtgcagggtatatcagttgtggtaatgtccatggccacctttgcaagctccagatccaacttcgagaagctcagaggacccgtcataactttgtacggcggtcctaaataggtacgcactacagctacctattttgcagaagccgacagcaagtatctaaacactaatcagctacaatagtcagcatagtacatttcatctgactaatactacaacaccaccaccactagtaacttgacaattaagtatgaaggtatatgtgtcccctaagagacacactgtacatagcaaataatctatagatcaaagggctacgcaacccctgaatagtaacaaaatacaaaatcactaaaaattataaaaacagaaaaatacataaataggtatacgtgtcccctaagagacacattgtatgtaggtgataagtatagatcaaagggccgaataacccctgaatagtaacaaaatatgaaaatcaataaaaatcataaaatagaaaaaccataaacagaagtagttcaaagggctataaaacccctgaatagtaacaaaacataaagttaataaaaatcaaatgaataccataattggcaaacggaagagatgtaggtacttaagcttcctaaaagcagccgaactcactttgagaagtaggcatagcataccgaactcttccacgatttcgaaaccacac

SEQ ID NO: 3 - DNA, 8136 bp, Artificial sequence
  Synthetic construct
  misc_feature: CHIKV DRDE, containing DRDE 5′ UTR, S27 3′ UTR, and SpeI cloning site
atggctgcgtgagacacacgtagcctaccagtttcttactgctctactctgcaaagcaagagattaataacccatcatggatcctgtgtacgtggacatagacgctgacagcgcctttttgaaggccctgcaacgtgcgtaccccatgtttgaggtggaaccaaggcaggtcacaccgaatgaccatgctaatgctagagcgttctcgcatctagctataaaactaatagagcaggaaattgaccccgactcaaccatcctggatatcggcagtgcgccagcaaggaggatgatgtcggacaggaagtaccactgcgtctgcccgatgcgcagtgcggaagatcccgagagactcgctaattatgcgagaaagctagcatctgccgcaggaaaagtcctggacagaaacatctctggaaagatcggggacttacaagcagtaatggccgtgccagacaaggagacgccaacattctgcttacacacagacgtctcatgtagacagagagcagacgtcgctatataccaagacgtctatgctgtacacgcacccacgtcgctataccaccaggcgattaaaggggtccgagtggcgtactgggttgggttcgacacaaccccgttcatgtacaatgccatggcgggtgcctacccctcatactcgacaaactgggcagatgagcaggtactgaaggctaagaacataggattatgttcaacagacctgacggaaggtagacgaggcaagttgtctattatgagagggaaaaagctaaaaccgtgcgaccgtgtgctgttctcagtagggtcaacgctctacccggaaagccgcaagctacttaagagctggcacctgccatcggtgttccatttaaagggcaaactcagcttcacatgccgctgtgatacagtggtttcgtgtgagggctacgtcgttaagagaataacgatgagcccaggcctttatggaaaaaccacagggtatgcggtaacccaccacgcagacggattcctgctgtgcaagactaccgacacggttgacggcgaaagagtgtcattctcggtgtgcacatacgtgccggcgaccatttgtgatcaaatgaccggcatccttgctacagaagtcacgccggaggatgcacagaagctgttggtggggctgaaccagagaatagtggttaacggcagaacgcaacggaatatgaacaccatgaaaaattatctgcttcccgtggtcgcccaagccttcagtaagtgggcaaaggagtgccggaaagacatggaagatgaaaaactcctgggggtcagagaaagaacactgacctgctgctgtctatgggcattcaagaagcagaaaacacacacggtctacaagaggcctgatacccagtcaattcagaaggttcaggccgagtttgacagctttgtggtaccgagtctgtggtcgtccgggttgtcaatccctttgaggactagaatcaaatggttgttaagcaaggtgccaaaaaccgacctgatcccatacagcggagacgcccgagaagcccgggacgcagaaaaagaagcagaggaagaacgagaagcagaactgactcgcgaagccctaccacctctacaggcagcacaggaagatgttcaggtcgaaatcgacgtggaacagcttgaggacagagcgggcgcaggaataatagagactccgagaggagctatcaaagttactgcccaaccaacagaccacgtcgtgggagagtacctggtactctccccgcagaccgtactacgtagccagaagctcagtctgattcacgctttggcggagcaagtgaagacgtgcacgcacaacggacgagcagggaggtatgcggtcgaagcgtacgacggccgagtcctagtgccctcaggctatgcaatctcgcctgaagacttccagagtctaagcgaaagcgcgacgatggtgtataacgaaagagagttcgtaaacagaaagctacaccatattgcgatgcacggaccagccctgaacaccgacgaagagtcgtatgagctggtgagggcagagaggacagaacacgagtacgtctacgacgtggatcagagaagatgctgtaagaaggaagaagccgcaggactggtactggtgggcgacttgactaatccgccctaccacgaattcgcatatgaagggctaaaaatccgccctgcctgcccatacaaaattgcagtcataggagtcttcggagtaccgggatctggcaagtcagctattatcaagaacctagttaccaggcaggacctggtgactagcggaaagaaagaaaactgccaagaaatcaccaccgacgtgatgagacagagaggtctagagatatctgcacgtacggttgactcgctgctcttgaatggatgcaacagaccagtcgacgtgttgtacgtagacgaggcgtttgcgtgccactctggaacgctacttgctttgatcgccttggtgagaccaaggcagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgaccgccattgtgtcatcgttgcattacgaaggcaaaatgcgcactacgaatgagtacaacaagccgatcgtagtggacactacaggctcaacaaaacctgaccctggagacctcgtgttaacgtgcttcagagggtgggttaaacaactgcaaattgactatcgtggatacgaggtcatgacagcagccgcatcccaagggttaaccagaaaaggagtttacgcagttagacaaaaagttaatgaaaacccgctctatgcatcaacgtcagagcacgtcaacgtactcctaacgcgtacggaaggtaaactggtatggaagacactttccggcgacccgtggataaagacgctgcagaacccaccgaaaggaaacttcaaagcaactattaaggagtgggaggtggagcatgcatcaataatggcgggcatctgcagtcaccaaatgaccttcgatacattccaaaataaagccaacgtttgttgggctaagagcttggtccctatcctcgaaacagcggggataaaactaaatgataggcagtggtctcagataattcaagccttcaaagaagacaaagcatactcacctgaagtagccctgaatgaaatatgtacgcgcatgtatggggtggatctagacagcgggctattttctaaaccgttggtgtctgtgtattacgcggataaccactgggataataggcctggagggaaaatgttcggatttaaccccgaggcagcatccattctagaaagaaagtatccattcacaaaagggaagtggaacatcaacaagcagatctgcgtgactaccaggaggatagaagactttaaccctaccaccaacatcataccggccaacaggagactaccacactcattagtggccgaacaccgcccagtaaaaggggaaagaatggaatggctggttaacaagataaacggccaccacgtgctcctggtcagtggctataaccttgcactgcctactaagagagtcacttgggtagcgccgttaggtgtccgcggagcggactacacatacaacctagagttgggtctgccagcaacgcttggtaggtatgaccttgtggtcataaacatccacacaccttttcgcatacaccattaccaacagtgcgtcgaccacgcaatgaaactgcaaatgctcgggggtgactcattgagactgctcaaaccgggcggctctctattgatcagagcatatggttacgcagatagaaccagtgaacgagtcatctgcgtattgggacgcaagtttagatcgtctagagcgttgaaaccaccatgtgtcaccagcaacactgagatgtttttcctattcagcaactttgacaatggcagaaggaatttcacaactcatgtcatgaacaatcaactgaatgcagccttcgtaggacaggtcacccgagcaggatgtgcaccgtcgtaccgggtaaaacgcatggacatcgcgaagaacgatgaagagtgcgtagtcaacgccgctaaccctcgcgggttaccgggtgacggtgtttgcaaggcagtatacaaaaaatggccggagtcctttaagaacagtgcaacaccagtgggaaccgcaaaaacagttatgtgcggtacgtatccagtaatccacgctgttggaccaaacttctctaattattcggagtctgaaggggaccgggaattggcagctgcctatcgagaagtcgcaaaggaagtaactaggctgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtagagctgctggatgagcacatctccatagactgcgatattgttcgcgtgcaccctgacagcagcttggcaggcagaaaaggatacagcaccacggaaggcgcactgtactcatatctagaagggacccgttttcatcagacggctgtggatatggcggagatacatactatgtggccaaagcaaacagaggccaatgagcaagtctgcctatatgccctgggggaaagtattgaatcgatcaggcagaaatgcccggtggatgatgcagacgcatcatctccccccaaaactgtcccgtgcctttgccgttacgctatgactccagaacgcgtcacccggcttcgcatgaaccacgtcacaagcataattgtgtgttcttcgtttcccctcccaaagtacaaaatagaaggagtgcaaaaagtcaaatgctctaaggtaatgctatttgaccacaacgtgccatcgcgcgtaagtccaagggaatatagatcttcccaggagtctgcacaggaggcgagtacaatcacgtcactgacgcatagtcaattcgacctaagcgttgatggcgagatactgcccgtcccgtcagacctggatgctgacgccccagccctagaaccagcactagacgacggggcgacacacacgctgccatccacaaccggaaaccttgcggccgtgtctgactgggtaatgagcaccgtacctgtcgcgccgcccagaagaaggcgagggagaaacctgactgtgacatgtgacgagagagaagggaatataacacccatggctagcgtccgattctttagggcagagctgtgtccggtcgtacaagaaacagcggagacgcgtgacacagcaatgtctcttcaggcaccaccgagtaccgccacggaaccgaatcatccgccgatctccttcggagcatcaagcgagacgttccccattacatttggggacttcaacgaaggagaaatcgaaagcttgtcttctgagctactaactttcggagacttcttaccaggagaagtggatgacttgacagacagcgactggtccacgtgctcagacacggacgacgagttatgactagacagggcaggtgggtatatattctcgtcggacaccggtccaggtcatttacaacagaagtcagtacgccagtcagtgctgccggtgaacaccctggaggaagtccacgaggagaagtgttacccacctaagctggatgaagcaaaggagcaactattacttaagaaactccaggagagtgcatccatggccaacagaagcaggtatcagtcgcgcaaagtagaaaacatgaaagcagcaatcatccagagactaaagagaggctgtagactatacttaatgtcagagaccccaaaagtccctacttaccggactacatatccggcgcctgtgtactcgcctccgatcaacgtccgattgtccaatcccgagtccgcagtggcagcatgcaatgagttcttagctagaaactatccaactgtctcatcataccaaattaccgacgagtatgatgcatatctagacatggtggacgggtcggagagttgcctggaccgagcgacattcaatccgtcaaaactcaggagctacccgaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaagttcgcatgcaaccaagaatactgggaagaatttgctgccagccctattaggataacaactgagaatttagcaacctatgttactaaactaaaagggccaaaagcagcagcgctattcgcaaaaacccataatctactgccactacaggaagtaccaatggataggttcacagtagatatgaaaagggacgtgaaggtgactcctggtacaaagcatacagaggaaagacctaaggtgcaggttatacaggcggctgaacccttggcgacagcatacctatgtgggattcacagagagctggttaggaggctgaacgccgtcctcctacccaatgtacatacactatttgacatgtctgccgaggatttcgatgccatcatagccgcacactttaagccaggagacactgttttggaaacggacatagcctcctttgataagagccaagatgattcacttgcgcttactgctttgatgctgttagaggatttaggggtggatcactccctgctggacttgatagaggctgctttcggagagatttccagctgtcacctaccgacaggtacgcgcttcaagttcggcgccatgatgaaatcaggtatgttcctaactctgttcgtcaacacattgttaaacatcaccatcgccagccgagtgctggaagatcgtctgacaaaatccgcgtgcgcggccttcatcggcgacgacaacataatacatggagtcgtctccgatgaattgatggcagccagatgtgccacttggatgaacatggaagtgaagatcatagatgcagttgtatccttgaaagccccttacttttgtggagggtttatactgcacgatactgtgacaggaacagcttgcagagtggcagacccgctaaaaaggctttttaaactgggcaaaccgctagcggcaggtgacgaacaagatgaagatagaagacgagcgctggctgacgaagtgatcagatggcaacgaacagggctaattgatgagctggagaaagcggtatactctaggtacgaagtgcagggtatatcagttgtggtaatgtccatggccacctttgcaagctccagatccaacttcgagaagctcagaggacccgtcataactttgtacggcggtcctaaataggtacgcactacagctacctattttgcagaagccgacagcaagtatctaaacactaatcagctacaatagtcagcatagtacatttcatctgactaatactacaacaccaccaccactagtaacttgacgactaagcatgaaggtatatgtgtcccctaagagacacaccgtatatagctaataatctgtagatcaaagggctatataacccctgaatagtaacaaaatacaaaatcactaaaaattataaaaaaaaaaaaaaaaaaacagaaaaatatataaataggtatacgtgtcccctaagagacacattgtatgtaggtgataagtatagatcaaagggccgaacaacccctgaatagtaacaaaatataaaaattaataaaaatcataaaatagaaaaaccataaacagaagtagttcaaagggctataaaaacccctgaatagtaacaaaacataaaactaataaaaatcaaatgaataccataattggcaaacggaagagatgtaggtacttaagcttcctaaaagcagccgaactcactttgagatgtaggcatagcataccgaactcttccacgattctccgaacccacagggacgtaggagatgttattttgtttttaatatttc

SEQ ID NO: 4 - DNA, 8146 bp, Artificial sequence
  Synthetic construct
  misc_feature: SINV Girdwood; derived from Genbank # MF459683; contains a SpeI cloning site
attggcggcgtagtacacactattgaatcaaacagccgaccaattgcactaccatcacaatggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattaccagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgacgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagcacatttcatctgactaataccacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctactagtgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttc

SEQ ID NO: 5 - DNA, 76 bp, Artificial sequence
  Synthetic construct
atggctgcgtgagacacacgtagcctaccagtttcttactgctctactctgcaaagcaagagattaataacccatc

SEQ ID NO: 6 - DNA, 513 bp, Artificial sequence
  Synthetic construct
cttgacgactaagcatgaaggtatatgtgtcccctaagagacacaccgtatatagctaataatctgtagatcaaagggctatataacccctgaatagtaacaaaatacaaaatcactaaaaattataaaaaaaaaaaaaaaaaaacagaaaaatatataaataggtatacgtgtcccctaagagacacattgtatgtaggtgataagtatagatcaaagggccgaacaacccctgaatagtaacaaaatataaaaattaataaaaatcataaaatagaaaaaccataaacagaagtagttcaaagggctataaaaacccctgaatagtaacaaaacataaaactaataaaaatcaaatgaataccataattggcaaacggaagagatgtaggtacttaagcttcctaaaagcagccgaactcactttgagatgtaggcatagcataccgaactcttccacgattctccgaacccacagggacgtaggagatgttattttgtttttaatatttc

SEQ ID NO: 7 - DNA, 18 bp, Artificial sequence
  Synthetic construct
  misc_feature: Bacteriophage T7 RNA polymerase promoter
taatacgactcactatag

SEQ ID NO: 8 - DNA, 29 bp, Artificial sequence
  Synthetic construct
  misc_feature: T7 terminator sequence
aacccctctctaaacggaggggttttttt

SEQ ID NO: 9 - protein, 15 aa, Artificial sequence
  Synthetic construct
  misc_feature: CD4 peptide
KSSFFRNVVWLIKKN

SEQ ID NO: 10 - protein, 9 aa, Artificial sequence
  Synthetic construct
  misc_feature: CD8 peptide
IYSTVASSL

SEQ ID NO: 11 - protein, 25 aa, Artificial sequence
  Synthetic construct
  misc_feature: ESR1 K303R peptide
LWPSPLMIKRSKRNSLALSLTADQM

SEQ ID NO: 12 - protein, 25 aa, Artificial Sequence
  Synthetic construct
  misc_feature: ESR1 E380Q peptid
VDLTLHDQVHLLQCAWLEILMIGLV

SEQ ID NO: 13 - protein, 23 aa, Artificial sequence
  Synthetic construct
  misc_feature: ESR1 Y537N peptide
SMKCKNVVPLNDLLLEMLDAHRL

SEQ ID NO: 14 - protein, 23 aa, Artificial sequence
  Synthetic construct
  misc_feature: ESR1 Y537S peptide
SMKCKNVVPLSDLLLEMLDAHRL

SEQ ID NO: 15 - protein, 23 aa, Artificial sequence
  Synthetic construct
  misc_feature: EESR1 Y537C peptide
SMKCKNVVPLCDLLLEMLDAHRL

SEQ ID NO: 16 - protein, 23 aa, Artificial sequence
  Synthetic construct
  misc_feature: ESR1 D538G peptide
SMKCKNVVPLYGLLLEMLDAHRL

(19) United States
(12) Patent Application Publication
(10) Pub. No.: US 2023/0398200 A1
(43) Pub. Date: Dec. 14, 2023

(54) MODIFIED CHIKUNGUNYA VIRUSES AND SINDBIS VIRUSES AND USES THEREOF

(71) Applicant: Replicate Bioscience, Inc., San Diego, CA (US)

(72) Inventors: Nathaniel Stephen WANG, San Diego, CA (US); Shigeki Joseph MIYAKE-STONER, San Diego, CA (US)

(21) Appl. No.: 18/007,036

(22) PCT Filed: Jul. 30, 2021
(86) PCT No.: PCT/US21/43991

§ 371 Date: Jan. 26, 2023

Related U.S. Application Data
(60) Provisional application No. 63/059,777, filed on Jul. 31, 2020.

(51) Int. Cl. A61K 39/12; C12N 15/86; C12N 15/88; A61K 45/06

(52) U.S. Cl. CPC A61K 39/12; C12N 15/86; C12N 15/88; A61K 45/06; A61K 2039/53; C12N 2770/36143; C12N 2770/36162; C12N 2770/36171; C12N 2770/36134

(57) ABSTRACT

The present disclosure relates to the field of molecular virology, including nucleic acid molecules comprising modified viral genomes or replicons, pharmaceutical compositions containing the same, and the use of such nucleic acid molecules and compositions for production of desired products in cell cultures or in a living body. Also provided are methods for eliciting an immune response in a subject in need thereof, as well as methods for preventing and/or treating various health conditions.

### FIELD

The present disclosure relates to the field of molecular virology and immunology, and particularly relates to nucleic acid molecules encoding modified viral genomes and replicons, pharmaceutical compositions containing the same, and the use of such nucleic acid molecules and compositions for production of desired products in cell cultures or in a living body. Also provided are methods for eliciting an immune response in a subject in need thereof, as well as methods for preventing and/or treating various health conditions.

### RELATED APPLICATION

This application claims priority to U.S. Provisional Patent Application Ser. No. 63/059,777, filed on Jul. 31, 2020, which is incorporated herein by reference in its entirety, including any drawings.

### INCORPORATION OF THE SEQUENCE LISTING

This application contains a Sequence Listing, which is hereby incorporated herein by reference in its entirety. The accompanying Sequence Listing text file, named “058462-501001WO_Sequence Listing_ST25.txt” was created on Jul. 15, 2021 and is 58 KB.

### BACKGROUND

In recent years, several different groups of animal viruses have been subjected to genetic manipulation either by homologous recombination or by direct engineering of their genomes. The availability of reverse genetics systems for both DNA and RNA viruses has created new perspectives for the use of recombinant viruses, for example, as vaccines, expression vectors, anti-tumor agents, gene therapy vectors, and drug delivery vehicles.

For example, many viral-based expression vectors have been deployed for expression of heterologous proteins in cultured recombinant cells. For example, the application of modified viral vectors for gene expression in host cells continues to expand. Recent advances in this regard include further development of techniques and systems for production of multi-subunit protein complexes, and co-expression of protein-modifying enzymes to improve heterologous protein production. Other recent progresses regarding viral expression vector technologies include many advanced genome engineering applications for controlling gene expression, preparation of viral vectors, in vivo gene therapy applications, and creation of vaccine delivery vectors.

However, it has been reported that host cells can develop intricate and powerful mechanisms to detect and counter pathogen invasion. It has been further reported that viruses, particularly pathogenic viruses, have evolved with host cells to combat these cellular defenses to infection and replication. As a result of infection, many host cells shut down cellular protein translation machinery in order to control viral replication and/or viral production of progeny that can potentially spread to additional cells. This phenomenon is generally termed as “innate immune response.” Infected cells also send out danger signals to other cells, locally and systemically, to set up an antiviral state and control the infection. Although these cellular antiviral systems benefit the host cells, they can also negatively impact self-amplifying RNAs (called replicons) designed to express beneficial vaccine antigens or therapeutic agents. For example, if a cell detects a replicon RNA expressing a beneficial protein and activates its innate immune defense mechanisms, the expression of the beneficial protein in such cell can be impacted and the efficacy of the replicon can be compromised.

Therefore, there is still a need for more efficient methods and systems for expressing products of interest in RNA replicon-based expression platforms.

### SUMMARY

The present disclosure relates generally to the development of immuno-therapeutics, such as recombinant nucleic acids constructs and pharmaceutical compositions including the same for use in the prevention and management of various health conditions such as proliferative disorders and microbial infection. In particular, as described in greater detail below, some embodiments of the disclosure provide nucleic acid constructs containing sequences that encode a modified genome or replicon of the alphavirus Chikungunya virus (CHIKV) or Sindbis virus (SINV) that is devoid at least a portion of the viral nucleic acid sequence encoding one or more structural proteins of the virus. Also disclosed are recombinant cells and transgenic animals that have been engineered to include one or more of the nucleic acid constructs disclosed herein, methods for producing a molecule of interest, pharmaceutical compositions including one or more of the following: (a) a nucleic acid construct of the disclosure, (b) a polypeptide of the disclosure, (c) a recombinant cell of the disclosure. Further provided in particular aspects of the disclosure are compositions and methods for eliciting an immune response in a subject in need thereof, and/or for the prevention and/or treatment of various health conditions, including proliferative disorders (e.g., cancers) and chronic infections.

In one aspect of the disclosure, provided herein are nucleic acid constructs including a nucleic acid sequence encoding a modified Chikungunya virus (CHIKV) genome or replicon RNA, wherein the modified CHIKV genome or replicon RNA is devoid of at least a portion of the nucleic acid sequence encoding one or more viral structural proteins.

In one aspect of the disclosure, provided herein are nucleic acid constructs including a nucleic acid sequence encoding a modified Sindbis virus (SINV) genome or replicon RNA, wherein the modified SINV genome or replicon RNA is devoid of at least a portion of the nucleic acid sequence encoding one or more viral structural proteins.

Non-limiting exemplary embodiments of the nucleic acid constructs of the disclosure can include one or more of the following features. In some embodiments, the modified viral genome or replicon RNA is devoid of a substantial portion of the nucleic acid sequence encoding one or more viral structural proteins. In some embodiments, the modified viral genome or replicon RNA includes no nucleic acid sequence encoding viral structural proteins.

In some embodiments, the nucleic acid molecules of the disclosure further include one or more expression cassettes, wherein each of the expression cassettes includes a promoter operably linked to a heterologous nucleic acid sequence. In some embodiments, at least one of the expression cassettes includes a subgenomic (sg) promoter operably linked to a heterologous nucleic acid sequence. In some embodiments, the sg promoter is a 26S subgenomic promoter. In some embodiments, the nucleic acid molecules of the disclosure further include one or more untranslated regions (UTRs). In some embodiments, at least one of the UTRs is a heterologous UTR.

In some embodiments, at least one of expression cassettes includes a coding sequence for a gene of interest (GOI). In some embodiments, the GOI encodes a polypeptide selected from the group consisting of a therapeutic polypeptide, a prophylactic polypeptide, a diagnostic polypeptide, a nutraceutical polypeptide, an industrial enzyme, and a reporter polypeptide. In some embodiments, the GOI encodes a polypeptide selected from the group consisting of an antibody, an antigen, an immune modulator, an enzyme, a signaling protein, and a cytokine. In some embodiments, the coding sequence of the GOI is optimized for expression at a level higher than the expression level of a reference coding sequence.

In some embodiments, the nucleic acid constructs of the disclosure include a nucleic acid sequence having at least 80%, at least 85%, at least 90%, at least 95%, at least 96%, at least 97%, at least 98%, at least 99%, or 100% sequence identity to a nucleic acid sequence selected from the group consisting of SEQ ID NOS: 1-4.

In one aspect, provided herein are recombinant cells including a nucleic acid construct as disclosed herein. In some embodiments, the recombinant cell is a eukaryotic cell. In some embodiments, the recombinant cell is an animal cell. In some embodiments, the animal cell is a vertebrate animal cell or an invertebrate animal cell. In some embodiments, the animal cell is an insect cell. In some embodiments, the insect cell is a mosquito cell. In some embodiments, the recombinant cell is a mammalian cell. In some embodiments, the recombinant cell is selected from the group consisting of a monkey kidney CV1 cell transformed by SV40 (COS-7), a human embryonic kidney cell (e.g., HEK 293 or HEK 293 cell), a baby hamster kidney cell (BHK), a mouse sertoli cell (e.g., TM4 cells), a monkey kidney cell (CV1), a human cervical carcinoma cell (HeLa), canine kidney cell (MDCK), buffalo rat liver cell (BRL 3A), human lung cell (W138), human liver cell (Hep G2), mouse mammary tumor (MMT 060562), TRI cell, FS4 cell, a Chinese hamster ovary cell (CHO cell), an African green monkey kidney cell (Vero cell), a human A549 cell, a human cervix cell, a human CHME5 cell, a human PER.C6 cell, a NS0 murine myeloma cell, a human epidermoid larynx cell, a human fibroblast cell, a human HUH-7 cell, a human MRC-5 cell, a human muscle cell, a human endothelial cell, a human astrocyte cell, a human macrophage cell, a human RAW 264.7 cell, a mouse 3T3 cell, a mouse L929 cell, a mouse connective tissue cell, a mouse muscle cell, and a rabbit kidney cell. Also provided, in a related aspect, are cell cultures that include at least one recombinant cell as disclosed herein and a culture medium.

In another aspect, provided herein are transgenic animals including a nucleic acid construct as described herein. In some embodiments, the transgenic animal is a vertebrate animal or an invertebrate animal. In some embodiments, the transgenic animal is a mammalian. In some embodiments, the transgenic mammalian is a non-human mammalian. In some embodiments, the transgenic animal is an insect. In some embodiments, the transgenic insect is a transgenic mosquito.

In another aspect, provided herein are methods for producing a polypeptide of interest (GOI), wherein the methods include (i) rearing an animal as disclosed herein, or (ii) culturing a recombinant cell including a nucleic acid construct as disclosed herein under conditions wherein the recombinant cell produces the polypeptide encoded by the GOI.

In another aspect, provided herein are methods for producing a polypeptide of interest in a subject, wherein the methods include administering to the subject a nucleic acid construct as disclosed herein. In some embodiments, the subject is vertebrate animal or an invertebrate animal. In some embodiments, the subject is an insect. In some embodiments, the subject is a mammalian subject. In some embodiments, the mammalian subject is a human subject. In yet another aspect, provided herein are recombinant polypeptides produced by a method of the disclosure.

In yet another aspect, provided herein are pharmaceutical compositions including a pharmaceutically acceptable excipient and: a) a nucleic acid construct of the disclosure; b) a recombinant cell of the disclosure; and/or c) a recombinant polypeptide of the disclosure.

Non-limiting exemplary embodiments of the pharmaceutical compositions of the disclosure can include one or more of the following features. In some embodiments, provided herein are compositions including a nucleic acid construct as disclosed herein and a pharmaceutically acceptable excipient. In some embodiments, provided herein are compositions including a recombinant cell as disclosed herein and a pharmaceutically acceptable excipient. In some embodiments, the compositions include a recombinant polypeptide of as disclosed herein and a pharmaceutically acceptable excipient. In some embodiments, provided herein are compositions that formulated in a liposome, a lipid-based nanoparticle (LNP), or a polymer nanoparticle. In some embodiments, the compositions are immunogenic compositions. In some embodiments, the immunogenic compositions are formulated as a vaccine. In some embodiments, the immunogenic compositions are substantially non-immunogenic to a subject. In some embodiments, the pharmaceutical compositions are formulated as an adjuvant. In some embodiments, the pharmaceutical compositions are formulated for one or more of intranasal administration, intranodal administration, transdermal administration, intraperitoneal administration, intramuscular administration, intratumoral administration, intraarticular administration, intravenous administration, subcutaneous administration, intravaginal administration, intraocular, oral, and rectal administration.

In another aspect, provided herein are methods for eliciting an immune response in a subject in need thereof, the method includes administering to the subject a composition including: a) a nucleic acid construct of the disclosure; b) a recombinant cell of the disclosure; c) a recombinant polypeptide of the disclosure; and/or d) a pharmaceutical composition of the disclosure.

In yet another aspect, provided herein are methods for preventing and/or treating a health condition in a subject in need thereof, the method includes prophylactically or therapeutically administering to the subject a composition including: a) a nucleic acid construct of the disclosure; b) a recombinant cell of the disclosure; c) a recombinant polypeptide of the disclosure; and/or d) a pharmaceutical composition of any one of the disclosure.

Non-limiting exemplary embodiments of the methods of the disclosure can include one or more of the following features. In some embodiments, the condition is a proliferative disorder or a microbial infection. In some embodiments, the subject has or is suspected of having a condition associated with proliferative disorder or a microbial infection. In some embodiments, the administered composition results in an increased production of interferon in the subject. In some embodiments, the composition is administered to the subject individually as a single therapy (monotherapy) or as a first therapy in combination with at least one additional therapies. In some embodiments, the at least one additional therapies is selected from the group consisting of chemotherapy, radiotherapy, immunotherapy, hormonal therapy, toxin therapy, targeted therapy, and surgery.

In yet another aspect, provided herein are kits for eliciting an immune response, for the prevention, and/or for the treatment of a health condition or a microbial infection, the kit including: a) a nucleic acid construct of the disclosure; b) a recombinant cell of the disclosure; c) a recombinant polypeptide of the disclosure; and/or d) a pharmaceutical composition of the disclosure.

Each of the aspects and embodiments described herein are capable of being used together, unless excluded either explicitly or clearly from the context of the embodiment or aspect.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 is a graphical representation of three non-limiting examples of the modified alphavirus genome designs in accordance with some embodiments of the disclosure, in which the nucleic acid sequence encoding viral structural proteins of the original virus have been completely deleted. Non-structural proteins nsP1, nsP2, nsP3, and nsP4 are shown. A non-limiting example of a modified CHIKV design be based on CHIKV strain S27 and further can contain a heterologous gene (GOI) placed under control of a 26S subgenomic promoter. A non-limiting example of a modified CHIKV design can also be based on CHIKV strain DRDE-06, contains the 3′ UTR derived from the CHIKV strain S27, and further can contain a heterologous gene (GOI) placed under control of a 26S subgenomic promoter. A non-limiting example of a modified SINV design can be based on SINV strain Girdwood and further can contain a heterologous gene (GOI) placed under control of a 26S subgenomic promoter

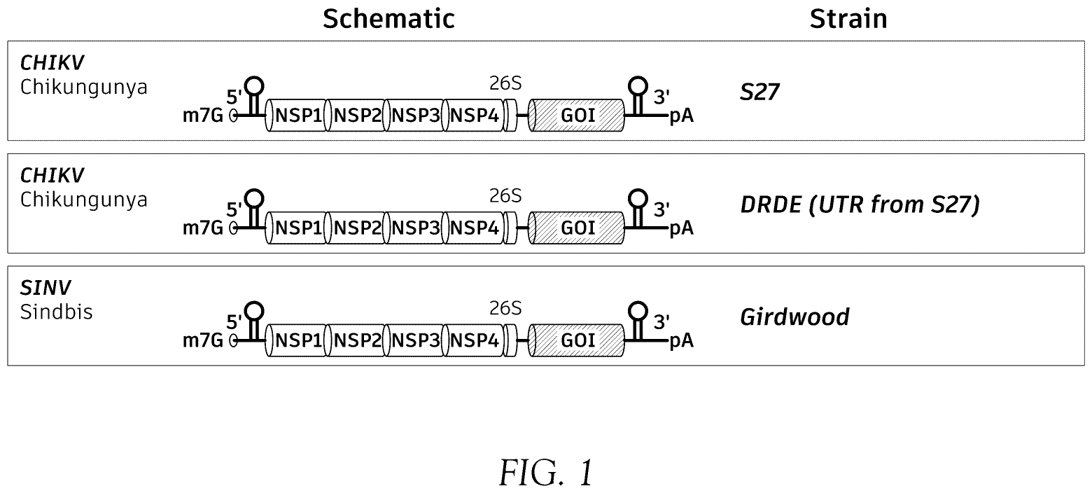

FIG. 2A is a graphical illustration of an exemplary alphavirus RNA replicon-based design pRB_008 Alpha-VEE-LAMP-HPV16 construct in accordance with some embodiments of the disclosure, in which the sequences encoding the modified VEEV is incorporated into expression vectors, which also include coding sequences for an exemplary gene of interest (GOI), e.g., human papillomavirus (HPV) oncoproteins E6/E7.

FIG. 2B is a graphical illustration of an exemplary alphavirus RNA replicon-based design pRB_009 Alpha-CHIKV-S27-LAMP-HPV16 construct in accordance with some embodiments of the disclosure, in which the sequences encoding the modified CHIKV S27 is incorporated into expression vectors, which also include coding sequences for an exemplary gene of interest (GOI), e.g., human papillomavirus (HPV) oncoproteins E6/E7.

FIG. 2C is a graphical illustration of an exemplary alphavirus RNA replicon-based design pRB_017 Alpha-CHIKV-DRDE-S27-LAMP-HPV16 construct in accordance with some embodiments of the disclosure, in which the sequences encoding the modified CHIKV DRDE is incorporated into expression vectors, which also include coding sequences for an exemplary gene of interest (GOI), e.g., human papillomavirus (HPV) oncoproteins E6/E7.

FIG. 2D is a graphical illustration of an exemplary alphavirus RNA replicon-based design pRB_017 Alpha-SIND-G-DLP-LAMP-HPV16 construct in accordance with some embodiments of the disclosure, in which the sequences encoding the modified SINV Girdwood genome is incorporated into expression vectors, which also include coding sequences for an exemplary gene of interest (GOI), e.g., human papillomavirus (HPV) oncoproteins E6/E7.

FIG. 3A is a graphical illustration of an exemplary alphavirus RNA replicon-based design VEE-HA construct in accordance with some embodiments of the disclosure, in which the sequence encoding the modified VEEV genome is incorporated into expression vectors, which also include coding sequences for an exemplary gene of interest (GOI), e.g., hemagglutinin precursor (HA) of the influenza A virus H5N1.

FIG. 3B is a graphical illustration of an exemplary alphavirus RNA replicon-based design CHIKV-S27-HA construct in accordance with some embodiments of the disclosure, in which the sequence encoding the modified CHIKV S27 genome is incorporated into expression vectors, which also include coding sequences for an exemplary gene of interest (GOI), e.g., hemagglutinin precursor (HA) of the influenza A virus H5N1.

FIG. 3C is a graphical illustration of an exemplary alphavirus RNA replicon-based design CHIKV-DRDE-HA construct in accordance with some embodiments of the disclosure, in which the sequence encoding the modified CHIKV DRDE genome is incorporated into expression vectors, which also include coding sequences for an exemplary gene of interest (GOI), e.g., hemagglutinin precursor (HA) of the influenza A virus H5N1.

FIG. 3D is a graphical illustration of an exemplary alphavirus RNA replicon-based design SIND-GW-HA construct in accordance with some embodiments of the disclosure, in which the sequence encoding the modified SINV Girdwood genome is incorporated into expression vectors, which also include coding sequences for an exemplary gene of interest (GOI), e.g., hemagglutinin precursor (HA) of the influenza A virus H5N1.

FIG. 3E is a graphical illustration of an exemplary alphavirus RNA replicon-based design VEE-Oncology construct in accordance with some embodiments of the disclosure, in which the sequence encoding the modified VEEV genome is incorporated into expression vectors, which also include coding sequences for an exemplary gene of interest (GOI), e.g., a synthetic sequence cassette encoding genes or parts of genes relevant to oncology (ESR1, HER2, and HER3).

FIG. 3F is a graphical illustration of an exemplary alphavirus RNA replicon-based design CHIKV-S27-Oncology construct in accordance with some embodiments of the disclosure, in which the sequence encoding the modified CHIKV S27 genome is incorporated into expression vectors, which also include coding sequences for an exemplary gene of interest (GOI), e.g., a synthetic sequence cassette encoding genes or parts of genes relevant to oncology (ESR1, HER2, and HER3).

FIG. 3G is a graphical illustration of an exemplary alphavirus RNA replicon-based design CHIKV-DRDE-Oncology construct in accordance with some embodiments of the disclosure, in which the sequence encoding the modified CHIKV DRDE genome is incorporated into expression vectors, which also include coding sequences for an exemplary gene of interest (GOI), e.g., a synthetic sequence cassette encoding genes or parts of genes relevant to oncology (ESR1, HER2, and HER3).

FIG. 3H is a graphical illustration of an exemplary alphavirus RNA replicon-based design SIND-GW-Oncology construct in accordance with some embodiments of the disclosure, in which the sequence encoding the modified SINV Girdwood genome is incorporated into expression vectors, which also include coding sequences for an exemplary gene of interest (GOI), e.g., a synthetic sequence cassette encoding genes or parts of genes relevant to oncology (ESR1, HER2, and HER3).

FIG. 4 is a graph illustrating the activity of an exemplary expressed transgene from CHIKV- or SINV-derived vectors encoding red firefly luciferase, demonstrating these vectors are capable of RNA replication and subsequent expression of transgenes that exhibit biological function. Vectors described in Examples 1 and 2 are used to prepare replicon RNA by IVT and transformed into BHK-21 cells in duplicate. At 18-20 h post transfection, enzymatic activity of red firefly luciferase produced by the transformed cells is quantified by the Luciferase Assay System protocol (Promega). RLU: Relative light units.

FIG. 5A graphically illustrates that antigen-specific T cell responses are differently affected by vector backbones (Day 14 post-prime with each vector). In vivo administration of the CHIKV- and SINV-derived vectors encoding HA antigen from H5N1 in BALB/c mice generates antigen-specific CD4+ and CD8+ T cell and functional antibody responses. CHIKV- and SINV- can be advantaged or disadvantaged for production of T cell responses compared with VEE-derived vectors, thus demonstrating their utility as vectors for vaccines or biotherapeutics. Geometric mean with geometric SD. One-way ANOVA.

FIG. 5B graphically illustrates neutralizing antibody responses post-immunization with each vector. HAI titers were measured Day 14 post-prime (left panel) or post-boost (right panel) with each vector. In vivo administration of the CHIKV- and SINV-derived vectors encoding HA antigen from H5N1 in BALB/c mice generates antigen-specific CD4+ and CD8+ T cell and functional antibody responses. CHIKV- and SINV- can be advantaged or disadvantaged for production of T cell responses compared with VEE-derived vectors, thus demonstrating their utility as vectors for vaccines or biotherapeutics. SFU: Spot forming units. Geometric mean with geometric SD. One-way ANOVA.

FIG. 6 is a graph illustrating vector-dependent differential T cell responses (Day 14 post boost with each vector) are induced by some, not all, antigens. In vivo administration of the CHIKV- and SINV-derived vectors encoding activating mutations from ESR1 and PI3K alongside truncated HER2 and kinase-dead HER3 proteins in BALB/c mice generate robust T cell responses. Responses varied by individual antigen encoded as well as by each vector and thus advantaged or disadvantaged in production of T cell responses compared with VEE-derived vectors, demonstrating their utility as vectors for vaccines or biotherapeutics. Geometric mean with geometric SD. One-way ANOVA.

### DETAILED DESCRIPTION OF THE DISCLOSURE

Provided herein are, inter alia, viral expression systems with superior expression potential which are suitable for expressing heterologous molecules such as, for example, vaccines and therapeutic polypeptides, in recombinant cells. For example, some embodiments of the disclosure relate to nucleic acid constructs such as, e.g. expression constructs and vectors, containing a modified genome or replicon RNA of a Chikungunya virus (CHIKV) or Sindbis virus (SINV) in which at least some of its original viral sequence encoding structural proteins has been deleted. Also provided in some embodiments of the disclosure are viral-based expression vectors including one or more expression cassettes encoding heterologous polypeptide. Further provided are recombinant cells that are genetically engineered to include one or more of the nucleic acid molecules disclosed herein. Biomaterials and recombinant products derived from such recombinant cells are also within the scope of the application. Also provided are compositions and methods useful for eliciting an immune response in a subject in need thereof, as well as methods for preventing and/or treating various health conditions.

Self-amplifying RNAs (replicons) based on RNA viruses (e.g., alphaviruses) can be used as robust expression systems. For example, it has been reported that an advantage of using alphaviruses such as CHIKV and SINV as viral expression vectors is that they can direct the synthesis of large amounts of heterologous proteins in recombinant host cells. Among other advantages, polypeptides such as therapeutic single chain antibodies can be most effective if expressed at high levels in vivo. In addition, for producing recombinant antibodies purified from cells in culture (ex vivo), high protein expression from a replicon RNA can increase overall yields of the antibody product. Furthermore, if the protein being expressed is a vaccine antigen, high level expression can induce the most robust immune response in vivo.

Alphaviruses utilize motifs contained in their UTRs, structural regions, and non-structural regions to impact their replication in host cells. These regions also contain mechanism to evade host cell innate immunity. However, significant differences among alphavirus species have been reported. For example, New World and Old World Alphaviruses have evolved different components to exploit stress granules, JAK-STAT signaling, FXR, and G3BP proteins within cells for assembly of viral replication complexes. Which part of the genome contains these components also varies between Alphaviruses. For example, bypassing activation of PKR and subsequent phosphorylation of EIF2alpha is done via the downstream loop in some Old World Alphaviruses such as Sindbis, but bypassing this pathway is thought to be done via NSP4 in Chikungunya, which lacks a recognizable DLP. In addition, beyond variation between individual Alphaviruses, there are often differences within strains of Alphaviruses as well that can account for changes in characteristics such as virulence. For example, sequence variations between North American and South American strains of Eastern Equine Encephalitis Virus (EEEV) alter the ability to modulate the STAT1 pathway leading to differential induction of Type I interferons and resulting changes in virulence.

Given the differential presence of host cell attenuating factors in non-structural and structural regions of Alphaviruses, deleting the structural genes to allow for heterologous gene expression in synthetic vectors will have varied impacts on individual vectors. Synthetic replicons with different host attenuating factors in the non-structural regions will differentially excel at the induction of immune responses to heterologous genes that are expressed. Chikungunya's ability to bypass PKR activation and subsequent EIF2alpha phosphorylation through motifs retained in the NSPs regions, robust activation of Type I interferons, and ability to exploit stress granules, JAK-STAT signaling, and G3BP proteins make it advantaged for use as a vaccine vector. Conversely, the avirulent Sindbis Girdwood strains inability to inhibit STAT1 makes it an advantaged vector for expression of heterologous proteins without forming robust immune responses against the encoded protein. As a further example, SINV strain S.A.AR86 (AR86) rapidly and robustly inhibits tyrosine phosphorylation of STAT1 and STAT2 in response to IFN-γ and/or IFN-β. A unique threonine at position 538 in the AR86 nsP1 results in slower non-structural protein processing and delayed subgenomic RNA synthesis from the related SINV strain Girdwood, which contributes to an adult mouse neurovirulence phenotype and can be advantageous for the kinetics and yield of heterologous protein expression and contribute to a more robust immune response to a vaccine antigen expressed from AR86-based replicon vectors. The advantages that these individual vectors confer has been up until now completely unexplored and unpredicted.

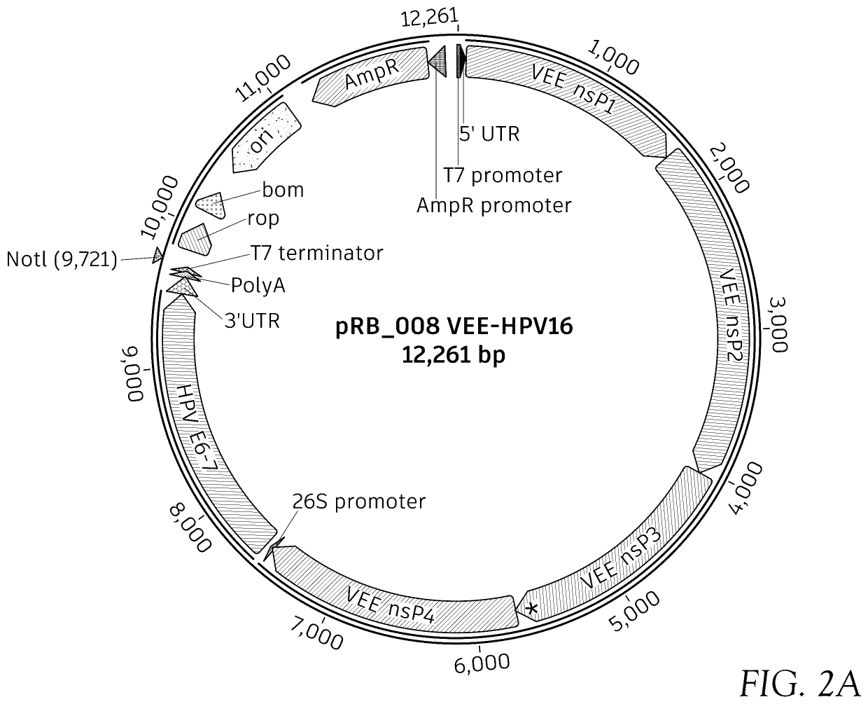

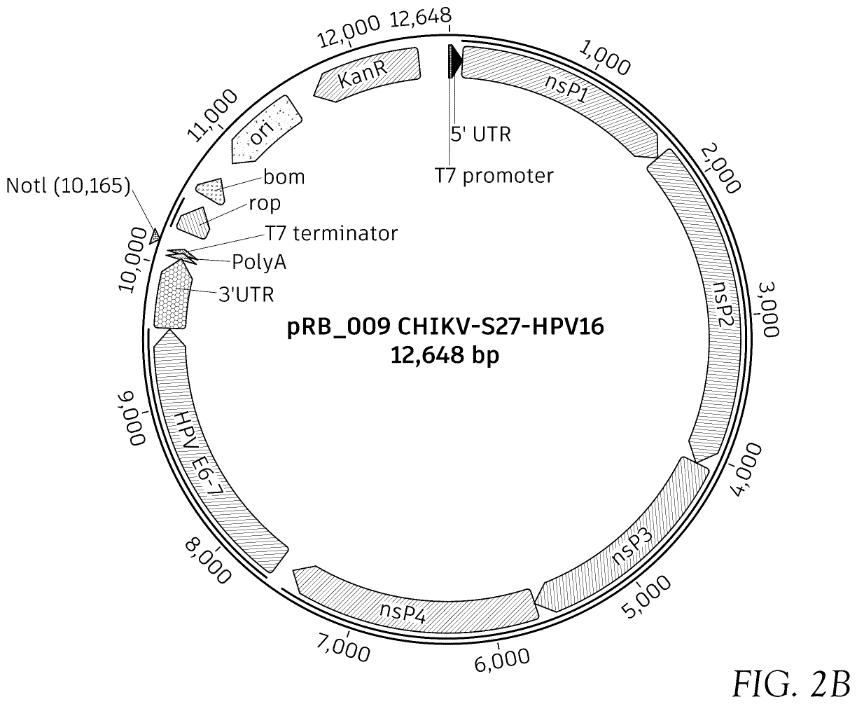

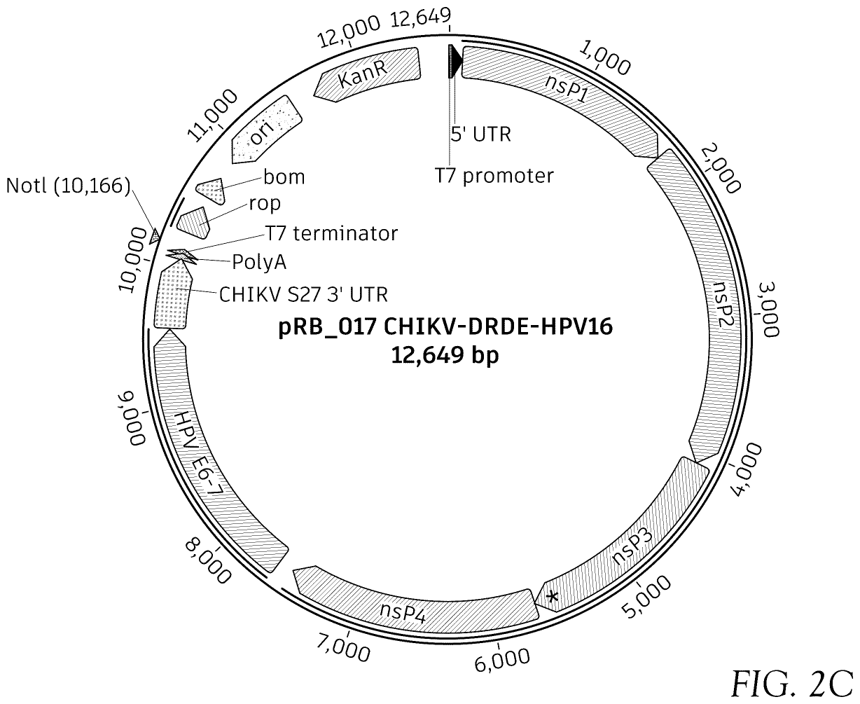

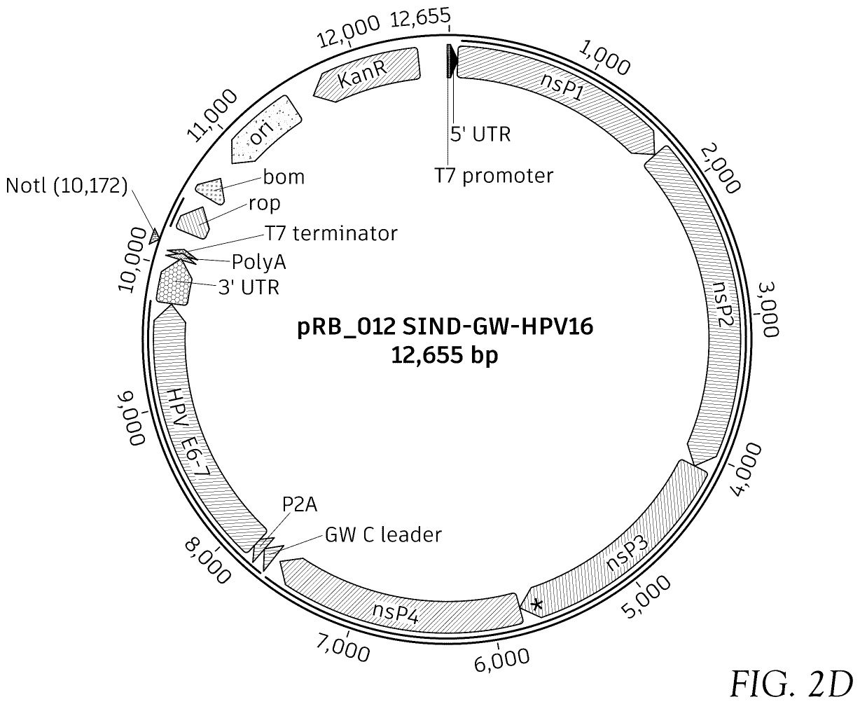

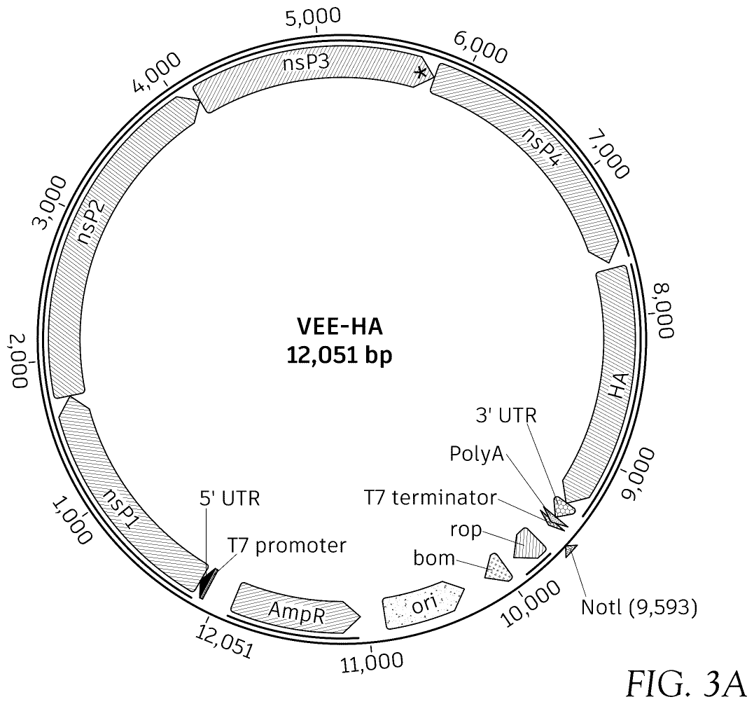

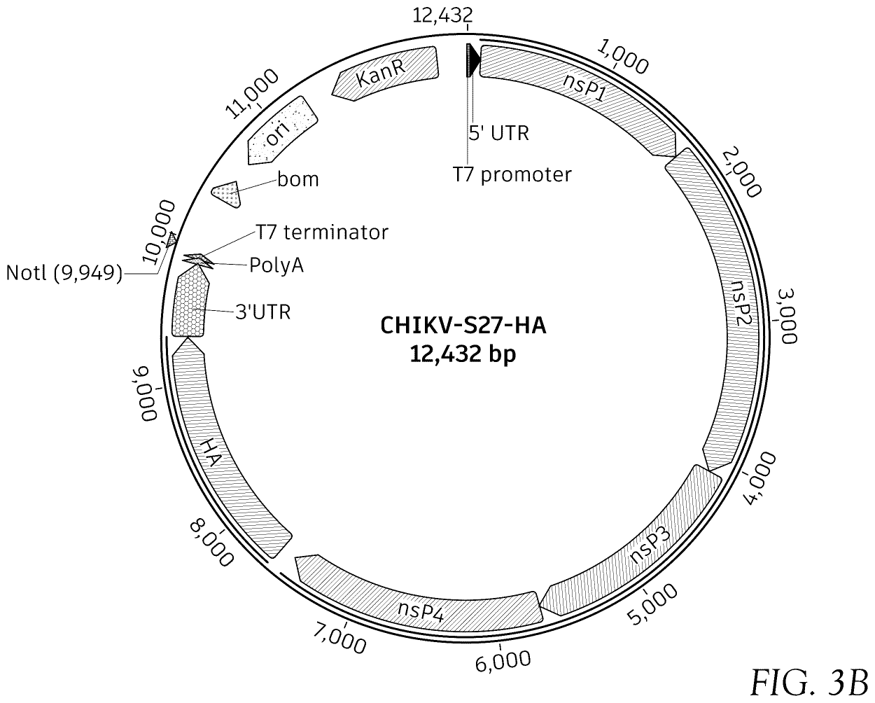

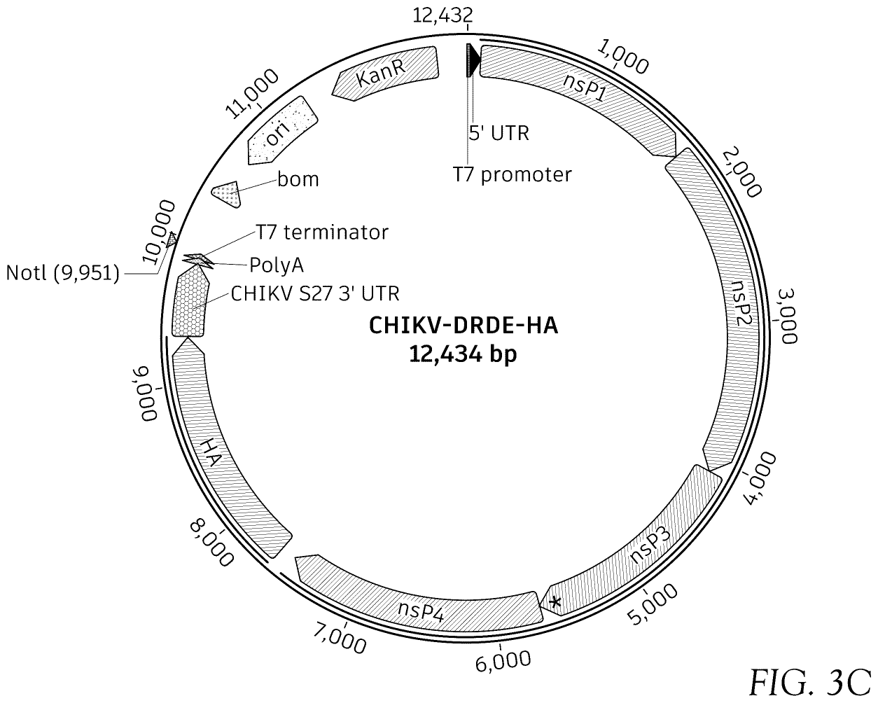

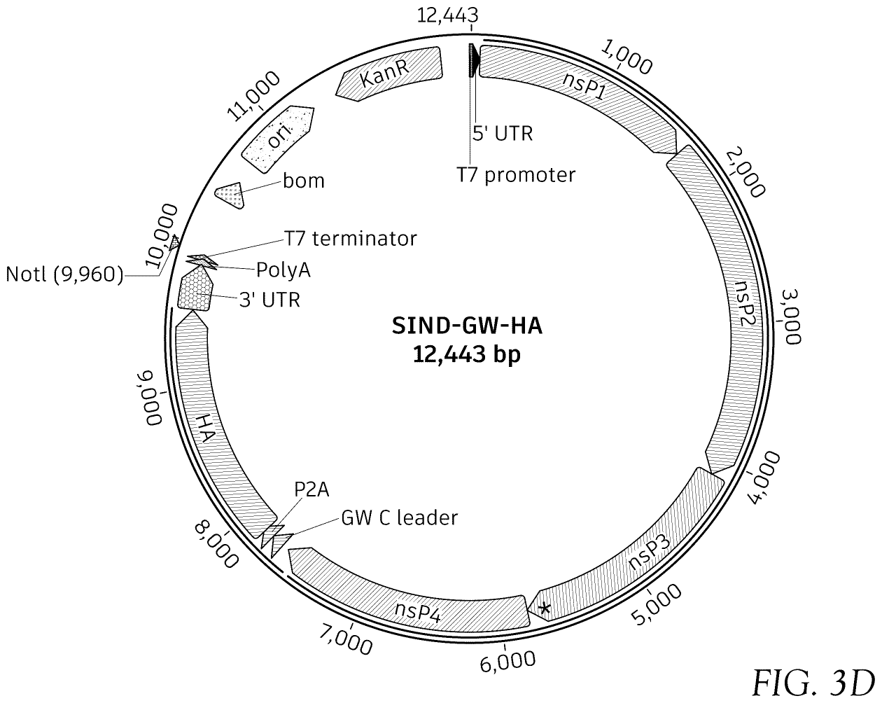

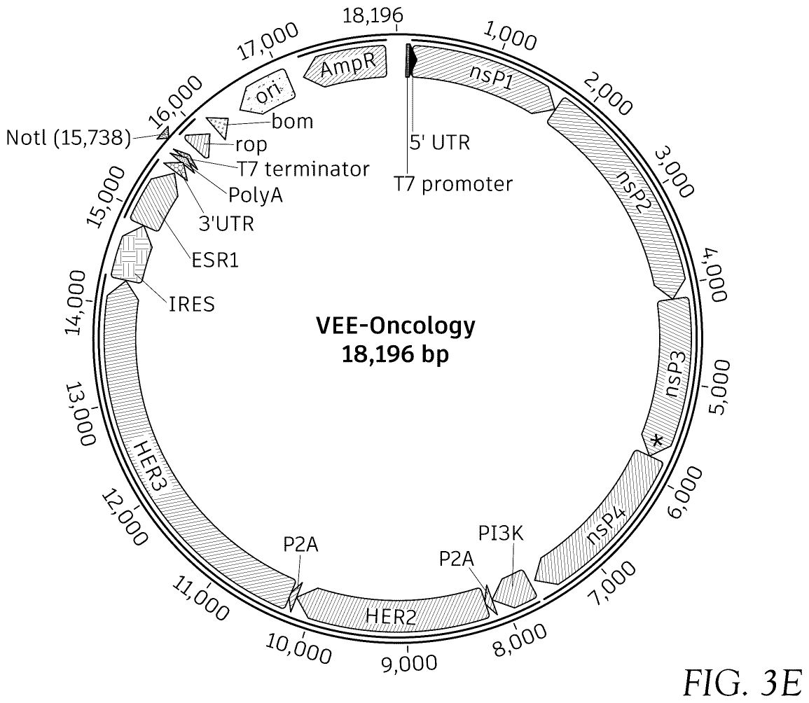

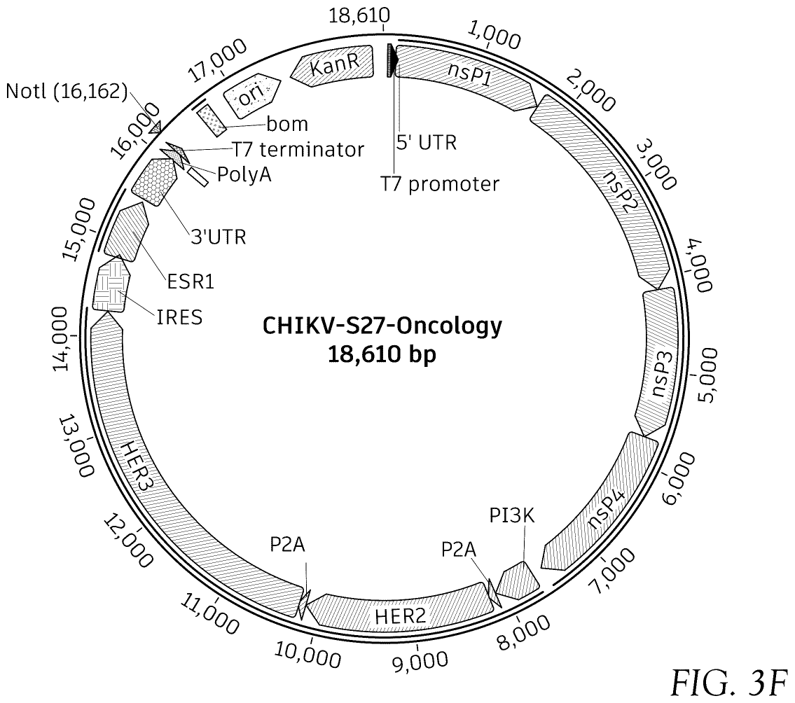

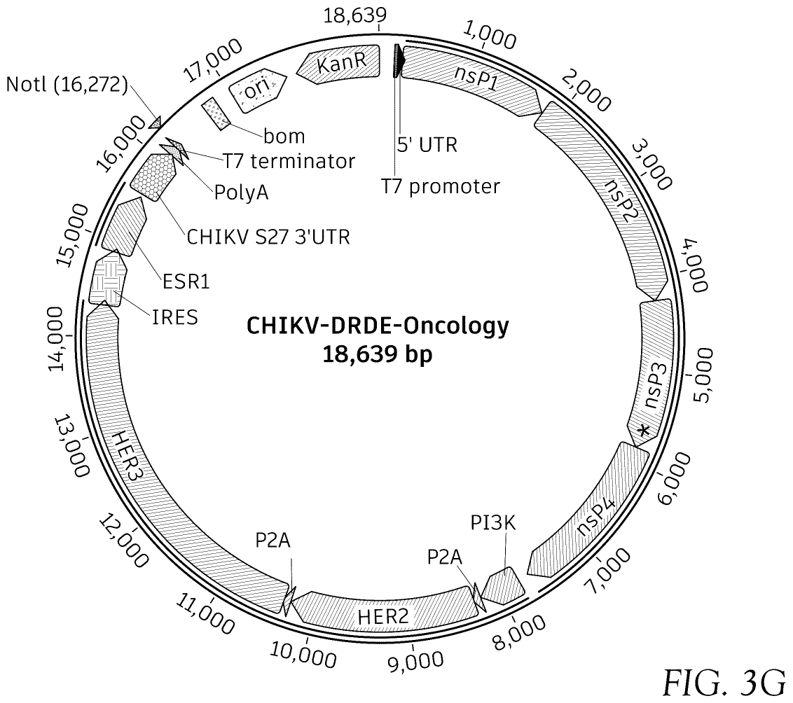

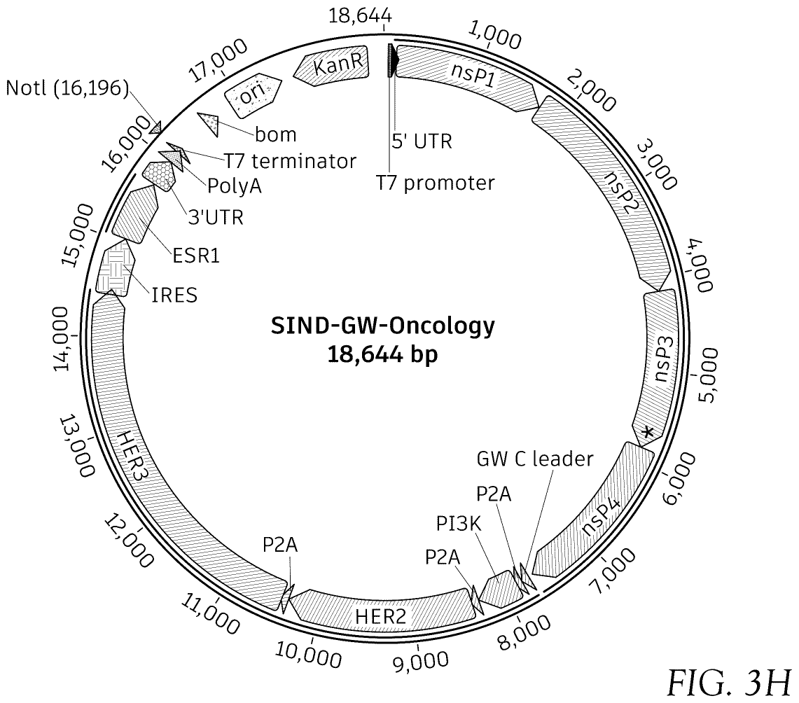

As described in greater detail below, an initial observation was made that the publicly available alphavirus genomic data does not always provide nucleotide sequences that are capable of direct replacement of the nucleic acid sequences encoding the structural proteins with a gene of interest (GOI) to result in self-replicating RNA and transgene-expressing replicons. For example, it was found possible to replace the structural polyprotein gene in CHIKV strain S27 (Genbank AF369024) with a synthetic HPV E6/E7 gene (human papillomavirus E6/E7 gene) (see, e.g., FIG. 2B) or hemagglutinin precursor (HA) of the influenza A virus H5N1 gene (see e.g., FIG. 3B) or red firefly luciferase gene to produce a replicon capable of RNA replication and transgene expression in transfected BHK-21 cells, however, the gene sequences used to similarly replace the structural polyprotein gene in CHIKV strain DRDE-06 (Genbank EF210157) were unable to undergo RNA replication or express the transgene. Thus, simple replacement of CHIKV structural proteins with heterologous genes using available, published sequences are not necessarily sufficient for generation of functional replicons. Stated differently, further engineering, such as using heterologous 5′ and/or 3′ UTR sequences, would be required to create replicon systems suitable for use in vaccines and therapeutics.

Notably, despite the numerous evolutionary divergences in sequence found across the genomes of CHIKV strains S27 and DRDE-06, the experimental data presented herein has demonstrated that a functional CHIKV strain DRDE replicon could be generated by replacing the DRDE 3′ UTR with the 3′ UTR from CHIKV strain S27 (see, e.g., FIG. 2C). Furthermore, the CHIKV and SINV replicon platform systems disclosed herein were found capable of expressing high levels of heterologous polypeptides of interest.

### Definitions

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof “A and/or B” is used herein to include all of the following alternatives: “A”, “B”, “A or B”, and “A and B”.

The terms “administration” and “administering”, as used herein, refer to the delivery of a bioactive composition or formulation by an administration route comprising, but not limited to, intranasal, transdermal, intravenous, intra-arterial, intramuscular, intranodal, intratumoral, intraarticular, intraperitoneal, subcutaneous, intramuscular, oral, rectal, intravaginal, intraocular, and topical administration, or combinations thereof. The term includes, but is not limited to, administering by a medical professional and self-administering.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications can occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “effective amount”, “therapeutically effective amount”, or “pharmaceutically effective amount” of a composition of the disclosure, e.g., nucleic acid constructs, recombinant cells, recombinant polypeptides, and/or pharmaceutical compositions, generally refers to an amount sufficient for the composition to accomplish a stated purpose relative to the absence of the composition (e.g., achieve the effect for which it is administered, stimulate an immune response, prevent or treat a disease, or reduce one or more symptoms of a disease, disorder, infection, or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges can independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about” which, as used herein, has its ordinary meaning of approximately. The term “about” is used to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value.

The term “construct” refers to a recombinant molecule including one or more isolated nucleic acid sequences from heterologous sources. For example, nucleic acid constructs can be chimeric nucleic acid molecules in which two or more nucleic acid sequences of different origin are assembled into a single nucleic acid molecule. Thus, representative nucleic acid constructs include any constructs that contain (1) nucleic acid sequences, including regulatory and coding sequences that are not found adjoined to one another in nature (e.g., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative nucleic acid constructs can include any recombinant nucleic acid molecules, linear or circular, single-stranded or double-stranded DNA or RNA nucleic acid molecules, derived from any source, such as a plasmid, cosmid, virus, autonomously replicating polynucleotide molecule, phage, capable of genomic integration or autonomous replication, comprising a nucleic acid molecule where one or more nucleic acid sequences have been operably linked. Constructs of the present disclosure can include the necessary elements to direct expression of a nucleic acid sequence of interest that is also contained in the construct. Such elements can include control elements such as a promoter that is operably linked to (so as to direct transcription of) the nucleic acid sequence of interest, and optionally includes a polyadenylation sequence. In some embodiments of the disclosure, the nucleic acid construct can be incorporated within a vector. In addition to the components of the construct, the vector can include, for example, one or more selectable markers, one or more origins of replication, such as prokaryotic and eukaryotic origins, at least one multiple cloning site, and/or elements to facilitate stable integration of the construct into the genome of a cell. Two or more constructs can be incorporated within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in a cell. For the practice of the present disclosure, compositions and methods for preparing and using constructs and cells are known to one skilled in the art.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the nucleic acid molecules described herein or the coding sequences and promoter sequences in a nucleic acid molecule means that the coding sequences and promoter sequences are in-frame and in proper spatial and distance away to permit the effects of the respective binding by transcription factors or RNA polymerase on transcription. It should be understood that operably linked elements can be contiguous or non-contiguous (e.g., linked to one another through a linker). In the context of polypeptide constructs, “operably linked” refers to a physical linkage (e.g., directly or indirectly linked) between amino acid sequences (e.g., different segments, portions, regions, or domains) to provide for a described activity of the constructs. Operably linked segments, portions, regions, and domains of the polypeptides or nucleic acid molecules disclosed herein can be contiguous or non-contiguous (e.g., linked to one another through a linker).

The term “portion” as used herein refers to a fraction. With respect to a particular structure such as a polynucleotide sequence or an amino acid sequence or protein the term “portion” thereof may designate a continuous or a discontinuous fraction of said structure. For example, a portion of an amino acid sequence comprises at least 1%, at least 5%, at least 10%, at least 20%, at least 30%, at least 40%, at least 50%, at least 60%, at least 70%, at least 80%, and at least 90% of the amino acids of said amino acid sequence. In addition or alternatively, if the portion is a discontinuous fraction, said discontinuous fraction is composed of 2, 3, 4, 5, 6, 7, 8, or more parts of a structure (e.g., domains of a protein), each part being a continuous element of the structure. For example, a discontinuous fraction of an amino acid sequence may be composed of 2, 3, 4, 5, 6, 7, 8, or more, for example not more than 4 parts of said amino acid sequence, wherein each part comprises at least 1, at least 2, at least 3, at least 4, at least 5 continuous amino acids, at least 10 continuous amino acids, at least 20 continuous amino acids, or at least 30 continuous amino acids of the amino acid sequence.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges can independently be included in the smaller ranges, and are also encompassed within the disclosure, subject to any specifically excluded limit in the stated range. Where the stated range includes one or both of the limits, ranges excluding either or both of those included limits are also included in the disclosure.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI website at ncbi.nlm.nih.gov/BLAST. Such sequences are then said to be “substantially identical.” This definition also refers to, or can be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “pharmaceutically acceptable excipient” as used herein refers to any suitable substance that provides a pharmaceutically acceptable carrier, additive, or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a health condition of interest (e.g., cancer or infection) and/or one or more symptoms of the health condition. The subject can also be an individual who is diagnosed with a risk of the health condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Chikungunya Virus (CHIKV) and Sindbis Virus (SINV)

Chikungunya virus (CHIKV) and Sindbis virus (SINV) are members of the genus Alphavirus which include a group of genetically, structurally, and serologically related viruses of the group IV Togaviridae family. Currently, the alphavirus genus includes among others the Sindbis virus (SINV), the Semliki Forest virus (SFV), the Ross River virus (RRV), Venezuelan equine encephalitis virus (VEEV), and Eastern equine encephalitis virus (EEEV), which are all closely related and are able to infect various vertebrates such as mammalians, rodents, fish, avian species, and larger mammals such as humans and horses as well as invertebrates such as insects. In particular, the Sindbis and the Semliki Forest viruses have been widely studied and the life cycle, mode of replication, etc., of these viruses are well characterized. SINV is a member of the Western Equine Encephalitis Virus Complex, whereas CHIKV is a member of the Semliki Forest virus complex and is closely related to Ross River virus, O′nyong′nyong virus, and Semliki Forest virus. In particular, alphaviruses have been shown to replicate very efficiently in animal cells which makes them valuable as vectors for production of protein and nucleic acids in such cells. Transmission between species and individuals occurs mainly via mosquitoes making the alphaviruses a contributor to the collection of Arboviruses—or Arthropod-Borne Viruses.

Each of these alphaviruses has a single stranded RNA genome of positive polarity enclosed in a nucleocapsid surrounded by an envelope containing viral spike proteins. Alphavirus particles are enveloped, tend to be spherical (although slightly pleomorphic), and have an isometric nucleocapsid. Alphavirus genome is single-stranded RNA of positive polarity of approximately 11-12 kb in length, comprising a 5′ cap, a 3′ poly-A tail, and two open reading frames with a first frame encoding the nonstructural proteins with enzymatic function and a second frame encoding the viral structural proteins (e.g., the capsid protein CP, E1 glycoprotein, E2 glycoprotein, E3 protein and 6K protein).

The 5′ two-thirds of the alphavirus genome encodes a number of nonstructural proteins necessary for transcription and replication of viral RNA. These proteins are translated directly from the RNA and together with cellular proteins form the RNA-dependent RNA polymerase essential for viral genome replication and transcription of subgenomic RNA. Four nonstructural proteins (nsP1-4) are produced as a single polyprotein constitute the virus' replication machinery. The processing of the polyprotein occurs in a highly regulated manner, with cleavage at the P2/3 junction influencing RNA template use during genome replication. This site is located at the base of a narrow cleft and is not readily accessible. Once cleaved, nsP3 creates a ring structure that encircles nsP2. These two proteins have an extensive interface. Mutations in nsP2 that produce noncytopathic viruses or a temperature sensitive phenotypes cluster at the P2/P3 interface region. P3 mutations opposite the location of the nsP2 noncytopathic mutations prevent efficient cleavage of P2/3. This in turn can affect RNA infectivity altering viral RNA production levels.

The 3′ one-third of the genome comprises subgenomic RNA which serves as a template for translation of all the structural proteins required for forming viral particles: the core nucleocapsid protein C, and the envelope proteins P62 and E1 that associate as a heterodimer. The viral membrane-anchored surface glycoproteins are responsible for receptor recognition and entry into target cells through membrane fusion. The subgenomic RNA is transcribed from the p26S subgenomic promoter present at the 3′ end of the RNA sequence encoding the nsP4 protein. The proteolytic maturation of P62 into E2 and E3 causes a change in the viral surface. Together the E1, E2, and sometimes E3, glycoprotein “spikes” form an E1/E2 dimer or an E1/E2/E3 trimer, where E2 extends from the center to the vertices, E1 fills the space between the vertices, and E3, if present, is at the distal end of the spike. Upon exposure of the virus to the acidity of the endosome, E1 dissociates from E2 to form an E1 homotrimer, which is necessary for the fusion step to drive the cellular and viral membranes together. The alphaviral glycoprotein E1 is a class II viral fusion protein, which is structurally different from the class I fusion proteins found in influenza virus and HIV. The E2 glycoprotein functions to interact with the nucleocapsid through its cytoplasmic domain, while its ectodomain is responsible for binding a cellular receptor. Most alphaviruses lose the peripheral protein E3, while in Semliki viruses it remains associated with the viral surface.

Alphavirus replication has been reported to take place on membranous surfaces within the host cell. In the first step of the infectious cycle, the 5′ end of the genomic RNA is translated into a polyprotein (nsP1-4) with RNA polymerase activity that produces a negative strand complementary to the genomic RNA. In a second step, the negative strand is used as a template for the production of two RNAs, respectively: (1) a positive genomic RNA corresponding to the genome of the secondary viruses producing, by translation, other nsP proteins and acting as a genome for the virus; and (2) subgenomic RNA encoding the structural proteins of the virus forming the infectious particles. The positive genomic RNA/subgenomic RNA ratio is regulated by proteolytic autocleavage of the polyprotein to nsP1, nsP2, nsP3 and nsP4. In practice, the viral gene expression takes place in two phases. In a first phase, there is main synthesis of positive genomic strands and of negative strands. During the second phase, the synthesis of subgenomic RNA is virtually exclusive, thus resulting in the production of large amount of structural protein.

### Compositions of the Disclosure

As described in greater detail below, one aspect of the present disclosure relates to nucleic acid constructs a nucleic acid sequence encoding a modified viral genome or replicon RNA, wherein the modified genome or replicon RNA is devoid of (e.g. does not include) at least a portion of the nucleic acid sequence encoding one or more structural proteins of the corresponding unmodified viral genome or replicon RNA. Some embodiments of the disclosure provide a modified alphavirus genome or replicon RNA in which the coding sequence for non-structural proteins nsP1, nsP2, nsP3, and nsP4 is present, however at least a portion of or the entire sequence encoding one or more structural proteins is absent. Also provided are recombinant cells and cell cultures that have been engineered to include a nucleic acid construct as disclosed herein.

### A. Nucleic Acid Constructs

As described in greater detail below, one aspect of the present disclosure relates to novel nucleic acid constructs including a nucleic acid sequence encoding a modified genome or replicon RNA of an alphavirus, such as Chikungunya virus (CHIKV) or Sindbis virus (SINV). For example, a modified alphavirus genome can include deletion(s), substitution(s), and/or insertion(s) in one or more of the genomic regions of the parent alphavirus genome.

Non-limiting exemplary embodiments of the nucleic acid constructs of the disclosure can include one or more of the following features. In some embodiments, the nucleic acid constructs include a nucleic acid sequence encoding a modified CHIKV genome or replicon RNA, wherein the modified CHIKV genome or replicon RNA is devoid of at least a portion of the nucleic acid sequence encoding one or more structural proteins of the unmodified CHIKV genome or replicon RNA, e.g., the modified CHIKV genome or replicon RNA does not include at least a portion of the coding sequence for one or more of the CHIKV structural proteins CP, E1, E2, E3, and 6K. Virulent and avirulent CHIKV strains are both suitable. Non-limiting examples of CHIKV strains suitable for the compositions and methods of the disclosure include CHIKV S27, CHIKV LR2006-OPY-1, CHIKV YO123223, CHIKV DRDE, CHIKV 37997, CHIKV 99653, CHIKV Ag41855, and Nagpur (India) 653496 strain. Additional examples of CHIKV strains suitable for the compositions and methods of the disclosure include, but are not limited to those described in Afreen et al. Microbiol. Immunol. 2014, 58:688-696, Lanciotti and Lambert ASTMH 2016, 94(4):800-803 and Langsjoen et al. mBio. 2018, 9(2): e02449-17. In some embodiments, the modified CHIKV genome or replicon RNA is derived from CHIKV strain S27 strain. In some embodiments, the modified CHIKV genome or replicon RNA is derived from CHIKV strain DRDE. In some embodiments, the modified CHIKV genome or replicon RNA is derived from CHIKV strain DRDE-06. In some embodiments, the modified CHIKV genome or replicon RNA is derived from CHIKV strain DRDE-07.

In some embodiments, the nucleic acid constructs include a nucleic acid sequence encoding a modified SINV genome or replicon RNA, wherein the modified SINV genome or replicon RNA is devoid of at least a portion of the nucleic acid sequence encoding one or more structural proteins of the unmodified SINV genome or replicon RNA, e.g., the modified CHIKV genome or replicon RNA does not include at least a portion of the coding sequence for one or more of the SINV structural proteins CP, E1, E2, E3, and 6K. Virulent and avirulent SINV strains are both suitable. Non-limiting examples of SINV strains suitable for the compositions and methods of the disclosure include SINV strain AR339 and Girdwood. Additional examples of SINV strains suitable for the compositions and methods of the disclosure include, but are not limited to those described in Sammels et al. J. Gen. Virol. 1999, 80(3):739-748, Lundström and Pfeffer Vector Borne Zoonotic Dis. 2010, 10(9):889-907, Sigei et al. Arch. of Virol. 2018, 163:2465-2469 and Ling et al. J. Virol. 2019, 93:e00620-19. In some embodiments, the modified SINV genome or replicon RNA is derived from SINV strain Girdwood. In some embodiments, the modified SINV genome or replicon RNA is derived from SINV strain AR86.

Non-limiting exemplary embodiments of the nucleic acid constructs of the disclosure can include one or more of the following features. In some embodiments, the modified viral genome or replicon RNA is devoid of at least a portion of the nucleic acid sequence encoding one or more of the viral structural proteins CP, E1, E2, E3, and 6K of the unmodified viral genome or replicon RNA. In some embodiments, the modified viral genome or replicon RNA is devoid of a portion of or the entire sequence encoding CP. In some embodiments, the modified viral genome or replicon RNA is devoid of a portion of or the entire sequence encoding E1. In some embodiments, the modified viral genome or replicon RNA is devoid of a portion of or the entire sequence encoding E2. In some embodiments, the modified viral genome or replicon RNA is devoid of a portion of or the entire sequence encoding E3. In some embodiments, the modified viral genome or replicon RNA is devoid of a portion of or the entire sequence encoding 6K. In some embodiments, the modified viral genome or replicon RNA is devoid of a portion of or the entire sequence encoding a combination of CP, E1, E2, E3, and 6K. Some embodiments of the disclosure provide a modified CHIKV genome or replicon RNA in which the coding sequence for non-structural proteins nsP1, nsP2, nsP3, and nsP4 of the unmodified CHIKV genome or replicon RNA is present, however at least a portion of or the entire sequence encoding one or more structural proteins (e.g., CP, E1, E2, E3, and 6K) of the CHIKV genome or replicon RNA is absent. Some embodiments of the disclosure provide a modified SINV genome or replicon RNA in which the coding sequence for non-structural proteins nsP1, nsP2, nsP3, and nsP4 of the unmodified SINV genome or replicon RNA is present, however at least a portion of or the entire sequence encoding one or more structural proteins (e.g., CP, E1, E2, E3, and 6K) of the SINV genome or replicon RNA is absent.

In some embodiments, the modified viral genome or replicon RNA is devoid of a substantial portion of the nucleic acid sequence encoding one or more viral structural proteins. The skilled artisan will understand that a substantial portion of a nucleic acid sequence encoding a viral structural polypeptide can include enough of the nucleic acid sequence encoding the viral structural polypeptide to afford putative identification of that polypeptide, either by manual evaluation of the sequence by one skilled in the art, or by computer-automated sequence comparison and identification using algorithms such as BLAST (see, for example, in “Basic Local Alignment Search Tool”; Altschul S F et al., J. Mol. Biol. 215:403-410, 1993). Accordingly, a substantial portion of a nucleotide sequence comprises enough of the sequence to afford specific identification and/or isolation of a nucleic acid fragment comprising the sequence. For example, a substantial portion of a nucleic acid sequence can include at least about 20%, for example, about 30%, about 40%, about 50%, about 60%, about 70%, about 80%, about 90%, about 95% of the full length nucleic acid sequence. As described above, the present disclosure provides nucleic acid molecules and constructs which are devoid of partial or complete nucleic acid sequences encoding one or more viral structural proteins. The skilled artisan, having the benefit of the sequences as disclosed herein, can readily use all or a substantial portion of the disclosed sequences for the compositions and methods of the disclosure. Accordingly, the present application comprises the complete sequences as disclosed herein, e.g., those set forth in the accompanying Sequence Listing, as well as substantial portions of those sequences as defined above.

In some embodiments, the modified viral genome or replicon RNA is devoid of the entire sequence encoding viral structural proteins, e.g., the modified viral genome or replicon RNA includes no nucleic acid sequence encoding the structural proteins of the viral unmodified genome or replicon RNA.

In some embodiments, the nucleic acid constructs of the disclosure further include one or more expression cassettes. In principle, the nucleic acid constructs disclosed herein can generally include any number of expression cassettes. In some embodiments, the nucleic acid constructs disclosed herein can include at least two, at least three, at least four, at least five, or at least six expression cassettes. The skilled artisan will understand that the term “expression cassette” refers to a construct of genetic material that contains coding sequences and enough regulatory information to direct proper transcription and/or translation of the coding sequences in a cell, in vivo and/or ex vivo. The expression cassette can be inserted into a vector for targeting to a desired host cell and/or into a subject. Accordingly, in some embodiments, the term expression cassette can be used interchangeably with the term “expression construct.” In some embodiments, the term “expression cassette” refers to a nucleic acid construct that includes a gene encoding a protein or functional RNA operably linked to regulatory elements such as, for example, a promoter and/or a termination signal, and optionally, any or a combination of other nucleic acid sequences that affect the transcription or translation of the gene.

In some embodiments, at least one of the expression cassettes includes a promoter operably linked to a heterologous nucleic acid sequence. Accordingly, the nucleic acid constructs as provided herein can find use, for example, as an expression vector that, when including a regulatory element (e.g., a promoter) operably linked to a heterologous nucleic acid sequence, can affect expression of the heterologous nucleic acid sequence. In some embodiments, at least one of the expression cassettes includes a subgenomic (sg) promoter operably linked to a heterologous nucleic acid sequence. In some embodiments, the sg promoter is a 26S subgenomic promoter. In some embodiments, the nucleic acid molecules of the disclosure further include one or more untranslated regions (UTRs). In some embodiments, at least one of the UTRs is a heterologous UTR. In some embodiments, at least one of the heterologous UTRs includes a sequence having at least 80%, at least 85%, at least 90%, at least 95%, at least 96%, at least 97%, at least 98%, at least 99%, or 100% sequence identity to the nucleic acid sequence of SEQ ID NO: 5. In some embodiments, at least one of the heterologous UTRs includes a sequence having at least 80%, at least 85%, at least 90%, at least 95%, at least 96%, at least 97%, at least 98%, at least 99%, or 100% sequence identity to the nucleic acid sequence of SEQ ID NO: 6.

In some embodiments, at least one of expression cassettes includes a coding sequence for a gene of interest (GOI). In some embodiments, the coding sequence of the GOI is optimized for a desired property. For example, in some embodiments, the coding sequence of the GOI is optimized for expression at a level higher than the expression level of a reference coding sequence. With respect to sequence-optimization of nucleotide sequences, degeneracy of the genetic code provides the possibility to substitute at least one base of the protein encoding sequence of a gene with a different base without causing the amino acid sequence of the polypeptide produced from the gene to be changed. Hence, the nucleic acid constructs of the present disclosure can also have any base sequence that has been changed from any polynucleotide sequence disclosed herein by substitution in accordance with degeneracy of the genetic code. References describing codon usage are readily publicly available. In some embodiments, polynucleotide sequence variants can be produced for a variety of reasons, e.g., to optimize expression for a particular host (e.g., changing codon usage in the alphavirus mRNA to those preferred by other organisms such as human, non-human primates, hamster, mice, or monkey). Accordingly, in some embodiments, the coding sequence of the GOI is optimized for expression in a target host cell through the use of codons optimized for expression. The techniques for the construction of synthetic nucleic acid sequences encoding GOI using preferred codons optimal for host cell expression may be determined by computational methods analyzing the commonality of codon usage for encoding native proteins of the host cell genome and their relative abundance by techniques well known in the art. The codon usage database (http://www.kazusa.or.jp/codon) may be used for generation of codon optimized sequences in mammalian cell environments. Furthermore, a variety of software tools are available to convert sequences from one organism to the optimal codon usage for a different host organism such as the JCat Codon Optimization Tool (www.jcat.de), Integrated DNA Technologies (IDT) Codon Optimization Tool (https://www.idtdna.com/CodonOpt) or the Optimizer online codon optimization tool (http://genomes.urv.es/OPTIMIZER). Such synthetic sequences may be constructed by techniques known in the art for the construction of synthetic nucleic acid molecules and may be obtained from a variety of commercial vendors.

In some embodiments, the coding sequence of the GOI is optimized for enhanced RNA stability and/or expression. The stability of RNA generally relates to the “half-life” of RNA. “Half-life” relates to the period of time which is needed to eliminate half of the activity, amount, or number of molecules. In the context of the present disclosure, the half-life of an RNA is indicative for the stability of said RNA. The half-life of RNA may influence the “duration of expression” of the RNA. Additional information regarding principles, strategies, and methods for use in enhancing RNA stability can be found at, for example, Leppek K. et al., Combinatorial optimization of mRNA structure, stability, and translation for RNA-based therapeutics. bioRxiv. (Preprint). Mar. 30, 2021. doi: 10.1101/2021.03.29.437587.

The polypeptide encoded by a GOI can generally be any polypeptide, and can be, for example a therapeutic polypeptide, a prophylactic polypeptide, a diagnostic polypeptide, a nutraceutical polypeptide, an industrial enzyme, and a reporter polypeptide. In some embodiments, the GOI encodes a polypeptide selected from the group consisting of an antibody, an antigen, an immune modulator, an enzyme, a signaling protein, and a cytokine.

In some embodiments, the nucleic acid constructs of the disclosure include a nucleic acid sequence encoding a modified CHIKV or a modified SINV having at least 80%, at least 85%, at least 90%, at least 95%, at least 96%, at least 97%, at least 98%, at least 99%, or 100% sequence identity to a nucleic acid sequence selected from the group consisting of SEQ ID NOs: 1-4. In some embodiments, the nucleic acid constructs of the disclosure include a nucleic acid sequence encoding a modified CHIKV or a modified SINV having at least 80%, at least 85%, at least 90%, at least 95%, at least 96%, at least 97%, at least 98%, at least 99%, or 100% sequence identity to the nucleic acid sequence of SEQ ID NO: 1. In some embodiments, the nucleic acid constructs of the disclosure include a nucleic acid sequence encoding a modified CHIKV or a modified SINV having at least 80%, at least 85%, at least 90%, at least 95%, at least 96%, at least 97%, at least 98%, at least 99%, or 100% sequence identity to the nucleic acid sequence of SEQ ID NO: 2. In some embodiments, the nucleic acid constructs of the disclosure include a nucleic acid sequence encoding a modified CHIKV or a modified SINV having at least 80%, at least 85%, at least 90%, at least 95%, at least 96%, at least 97%, at least 98%, at least 99%, or 100% sequence identity to the nucleic acid sequence of SEQ ID NO: 3. In some embodiments, the nucleic acid constructs of the disclosure include a nucleic acid sequence encoding a modified CHIKV or a modified SINV having at least 80%, at least 85%, at least 90%, at least 95%, at least 96%, at least 97%, at least 98%, at least 99%, or 100% sequence identity to the nucleic acid sequence of SEQ ID NO: 4.

Nucleic acid sequences having a high degree of sequence identity (e.g., at least 80%, at least 85%, at least 90%, at least 95%, at least 96%, at least 97%, at least 98%, at least 99%, or 100%) to a sequence of a modified CHIKV or modified SINV of interest can be identified and/or isolated by using the sequences identified herein (e.g., SEQ ID NOS: 1-4) or any others as they are known in the art, by genome sequence analysis, hybridization, and/or PCR with degenerate primers or gene-specific primers from sequences identified in the respective CHIKV or SINV genome.

### B. Recombinant Cells

The nucleic acid constructs of the present disclosure can be introduced into a host cell to produce a recombinant cell containing the nucleic acid molecule. Accordingly, prokaryotic or eukaryotic cells that contain a nucleic acid construct encoding a modified CHIKV or SINV genome as described herein are also features of the disclosure. In a related aspect, some embodiments disclosed herein relate to methods of transforming a cell which includes introducing into a host cell, such as an animal cell, a nucleic acid construct as provided herein, and then selecting or screening for a transformed cell. Introduction of the nucleic acid constructs of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, direct micro-injection, nanoparticle-mediated nucleic acid delivery, and the like.

In one aspect, some embodiments of the disclosure relate to recombinant cells, for example, recombinant animal cells that include a nucleic acid construct described herein. The nucleic acid construct can be stably integrated in the host genome, or can be episomally replicating, or present in the recombinant host cell as a mini-circle expression vector for a stable or transient expression. Accordingly, in some embodiments of the disclosure, the nucleic acid construct is maintained and replicated in the recombinant host cell as an episomal unit. In some embodiments, the nucleic acid construct is stably integrated into the genome of the recombinant cell. Stable integration can be completed using classical random genomic recombination techniques or with more precise genome editing techniques such as using guide RNA directed CRISPR/Cas9 or TALEN genome editing. In some embodiments, the nucleic acid construct present in the recombinant host cell as a mini-circle expression vector for a stable or transient expression.

In some embodiments, the recombinant cell is a prokaryotic cell, such as the bacterium E. coli, or a eukaryotic cell, such as an insect cell (e.g., a mosquito cell or a Sf21 cell), or mammalian cells (e.g., COS cells, NIH 3T3 cells, or HeLa cells). In some embodiments, the cell is in vivo, for example, a recombinant cell in a living body, e.g., cell of a transgenic subject. In some embodiments, the subject is a vertebrate animal or an invertebrate animal. In some embodiments, the subject is an insect. In some embodiments, the subject is a mammalian subject. In some embodiments, the mammalian subject is a human subject. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a vertebrate animal cell or an invertebrate animal cell. In some embodiments, the recombinant cell is a mammalian cell. In some embodiments, the recombinant cell is selected from the group consisting of a monkey kidney CV1 cell transformed by SV40 (COS-7), a human embryonic kidney cell (e.g., HEK 293 or HEK 293 cell), a baby hamster kidney cell (BHK), a mouse sertoli cell (e.g., TM4 cells), a monkey kidney cell (CV1), a human cervical carcinoma cell (HeLa), canine kidney cell (MDCK), buffalo rat liver cell (BRL 3A), human lung cell (W138), human liver cell (Hep G2), mouse mammary tumor (MMT 060562), TRI cell, FS4 cell, a Chinese hamster ovary cell (CHO cell), an African green monkey kidney cell (Vero cell), a human A549 cell, a human cervix cell, a human CHME5 cell, a human PER. C6 cell, a NS0 murine myeloma cell, a human epidermoid larynx cell, a human fibroblast cell, a human HUH-7 cell, a human MRC-5 cell, a human muscle cell, a human endothelial cell, a human astrocyte cell, a human macrophage cell, a human RAW 264.7 cell, a mouse 3T3 cell, a mouse L929 cell, a mouse connective tissue cell, a mouse muscle cell, and a rabbit kidney cell.

In some embodiments, the recombinant cell is an insect cell, e.g., cell of an insect cell line. In some embodiments, the recombinant cell is a Sf21 cell. Additional suitable insect cell lines include, but are not limited to, cell lines established from insect orders Diptera, Lepidoptera and Hemiptera, and can be derived from different tissue sources. In some embodiments, the recombinant cell is a cell of a lepidopteran insect cell line. In the past few decades, the availability of lepidopteran insect cell lines has increased at about 50 lines per decade. More information regarding available lepidopteran insect cell lines can be found in, e.g., Lynn D. E., Available lepidopteran insect cell lines. Methods Mol Biol. 2007; 388:117-38, which is herein incorporated by reference. In some embodiments, the recombinant cell is a mosquito cell, e.g., a cell of mosquito species within Anopheles (An.), Culex (Cx.) and Aedes (Stegomyia) (Ae.) genera. Exemplary mosquito cell lines suitable for the compositions and methods described herein include cell lines from the following mosquito species: Aedes aegypti, Aedes albopictus, Aedes pseudoscutellaris, Aedes triseriatus, Aedes vexans, Anopheles gambiae, Anopheles stephensi, Anopheles albimanus, Culex quinquefasciatus, Culex theileri, Culex tritaeniorhynchus, Culex bitaeniorhynchus, and Toxorhynchites amboinensis. Suitable mosquito cell lines include, but are not limited to, CCL-125, Aag-2, RML-12, C6/26, C6/36, C7-10, AP-61, A.t. GRIP-1, A.t. GRIP-2, UM-AVE1, Mos.55, Sua1B, 4a-3B, Mos.43, MSQ43, and LSB-AA695BB. In some embodiments, the mosquito cell is a cell of a C6/26 cell line.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### C. Transgenic Animals

Also provided, in another aspect, are transgenic animals including a nucleic acid construct as described herein. In some embodiments, the transgenic animal is a vertebrate animal or an invertebrate animal. In some embodiments, the transgenic animal is an insect. In some embodiments, the insect is a mosquito. In some embodiments, the transgenic animal is a mammalian. In some embodiments, the transgenic mammalian is a non-human mammalian. In some embodiments, the transgenic animal produces a protein of interest as described herein.

The transgenic non-human host animals of the disclosure are prepared using standard methods known in the art for introducing exogenous nucleic acid into the genome of a non-human animal. In some embodiments, the non-human animals of the disclosure are non-human primates. Other animal species suitable for the compositions and methods of the disclosure include animals that are (i) suitable for transgenesis and (ii) capable of rearranging immunoglobulin gene segments to produce an antibody response. Examples of such species include but are not limited to mice, rats, hamsters, rabbits, chickens, goats, pigs, sheep and cows. Approaches and methods for preparing transgenic non-human animals are known in the art. Exemplary methods include pronuclear microinjection, DNA microinjection, lentiviral vector mediated DNA transfer into early embryos and sperm-mediated transgenesis, adenovirus mediated introduction of DNA into animal sperm (e.g., in pig), retroviral vectors (e.g., avian species), somatic cell nuclear transfer (e.g., in goats). The state of the art in the preparation of transgenic domestic farm animals is reviewed in Niemann, H. et al. (2005) Rev. Sci. Tech. 24:285-298.

In some embodiments of the disclosure, the transgenic animal is a vertebrate animal or an invertebrate animal. In some embodiments, the animal is an insect. In some embodiments, the insect is a mosquito. In some embodiments, the animal is a mammalian subject. In some embodiments, the mammalian animal is a non-human animal. In some embodiments, the mammalian animal is a non-human primate. In some embodiments, the transgenic animals of the disclosure can be made using classical random genomic recombination techniques or with more precise techniques such as guide RNA-directed CRISPR/Cas genome editing, or DNA-guided endonuclease genome editing with NgAgo (Natronobacterium gregoryi Argonaute), or TALENs genome editing (transcription activator-like effector nucleases). In some embodiments, the transgenic animals of the disclosure can be made using transgenic microinjection technology and do not require the use of homologous recombination technology and thus are considered to be easier to prepare and select than approaches using homologous recombination. In another aspect, provided herein are methods for producing a polypeptide of interest, wherein the methods include (i) rearing a transgenic animal as disclosed herein; or (ii) culturing a recombinant cell including a nucleic acid construct as disclosed herein under conditions wherein the transgenic animal or the recombinant cell produces the polypeptide encoded by the GOI.

In another aspect, provided herein are methods for producing a polypeptide of interest, wherein the methods include (i) rearing a transgenic animal as disclosed herein; or (ii) culturing a recombinant cell including a nucleic acid construct as disclosed herein under conditions wherein the transgenic animal or the recombinant cell produces the polypeptide encoded by the GOI. In another aspect, provided herein are methods for producing a polypeptide of interest in a subject, wherein the methods include administering to the subject a nucleic acid construct as disclosed herein. In some embodiments, the subject is vertebrate animal or an invertebrate animal. In some embodiments, the subject is a mammalian subject. In some embodiments, the mammalian subject is a human subject. Accordingly, the recombinant polypeptides produced by the method disclosed herein are also within the scope of the disclosure.

Non-limiting exemplary embodiments of the disclosed methods for producing a recombinant polypeptide can include one or more of the following features. In some embodiments, the methods for producing a recombinant polypeptide of the disclosure further include isolating and/or purifying the produced polypeptide. In some embodiments, the methods for producing a polypeptide of the disclosure further include structurally modifying the produced polypeptide to increase half-life.

### D. Pharmaceutical Compositions

The nucleic acid constructs, recombinant cells, recombinant polypeptides of the disclosure can be incorporated into compositions, including pharmaceutical compositions. Such compositions generally include one or more of the nucleic acid constructs, recombinant cells, recombinant polypeptides described and provided herein, and a pharmaceutically acceptable excipient, e.g., carrier. In some embodiments, the compositions of the disclosure are formulated for the prevention, treatment, or management of a health condition such as an immune disease or a microbial infection. For example, the compositions of the disclosure can be formulated as a prophylactic composition, a therapeutic composition, or a pharmaceutical composition comprising a pharmaceutically acceptable excipient, or a mixture thereof. In some embodiments, the compositions of the present disclosure are formulated for use as a vaccine. In some embodiments, the compositions of the present application are formulated for use as an adjuvant.

Accordingly, in one aspect, provided herein are pharmaceutical compositions including a pharmaceutically acceptable excipient and: a) a nucleic acid construct of the disclosure; b) a recombinant cell of the disclosure; and/or c) a recombinant polypeptide of the disclosure.

Non-limiting exemplary embodiments of the pharmaceutical compositions of the disclosure can include one or more of the following features. In some embodiments, provided herein are compositions including a nucleic acid construct as disclosed herein and a pharmaceutically acceptable excipient. In some embodiments, provided herein are compositions including a recombinant cell as disclosed herein and a pharmaceutically acceptable excipient. In some embodiments, the compositions include a recombinant polypeptide of as disclosed herein and a pharmaceutically acceptable excipient.

In some embodiments, the compositions of the disclosure that formulated in a liposome. In some embodiments, the compositions of the disclosure that formulated in a lipid-based nanoparticle (LNP). In some embodiments, the compositions of the disclosure that formulated in a polymer nanoparticle. In some embodiments, the compositions are immunogenic compositions, e.g., composition that can stimulate an immune response in a subject. In some embodiments, the immunogenic compositions are formulated as a vaccine. In some embodiments, the pharmaceutical compositions are formulated as an adjuvant.

In some embodiments, the immunogenic compositions are substantially non-immunogenic to a subject, e.g. compositions that minimally stimulate an immune response in a subject. In some embodiments, the non-immunogenic or minimally immunogenic compositions are formulated as a biotherapeutic. In some embodiments, the pharmaceutical compositions are formulated for one or more of intranasal administration, transdermal administration, intraperitoneal administration, intramuscular administration, intranodal administration, intratumoral administration, intraarticular administration, intravenous administration, subcutaneous administration, intravaginal administration, intraocular, rectal, and oral administration.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In these cases, the composition should be sterile and should be fluid to the extent that easy syringability exists. It can be stable under the conditions of manufacture and storage, and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the composition is formulated for one or more of intranasal administration, transdermal administration, intramuscular administration, intranodal administration, intratumoral administration, intraarticular administration, intravenous administration, intraperitoneal administration, oral administration, intravaginal, intraocular, rectal, or intra-cranial administration. In some embodiments, the administered composition results in an increased production of interferon in the subject.

### Methods of the Disclosure

Administration of any one of the therapeutic compositions described herein, e.g., nucleic acid constructs, recombinant cells, recombinant polypeptides, and/or pharmaceutical compositions, can be used in the treatment of relevant health conditions, such as proliferative disorders (e.g., cancers) and chronic infections (e.g., viral infections). In some embodiments, the nucleic acid constructs, recombinant cells, recombinant polypeptides, and/or pharmaceutical compositions as described herein can be incorporated into therapeutic agents for use in methods of treating an individual who has, who is suspected of having, or who may be at high risk for developing one or more relevant health conditions or diseases. Exemplary health conditions or diseases can include, without limitation, cancers, immune diseases, gene therapy, gene replacement, cardiovascular diseases, age-related pathologies, acute infection, and chronic infection. In some embodiments, the individual is a patient under the care of a physician.

Accordingly, in one aspect, provided herein are methods for eliciting an immune response in a subject in need thereof, the method includes administering to the subject a composition including: a) a nucleic acid construct of the disclosure; b) a recombinant cell of the disclosure; c) a recombinant polypeptide of the disclosure; and/or d) a pharmaceutical composition of the disclosure.

In another aspect, provided herein are methods for preventing and/or treating a health condition in a subject in need thereof, the method includes prophylactically or therapeutically administering to the subject a composition including: a) a nucleic acid construct of the disclosure; b) a recombinant cell of the disclosure; c) a recombinant polypeptide of the disclosure; and/or d) a pharmaceutical composition of any one of the disclosure.

In some embodiments, the health condition is a proliferative disorder or a microbial infection. In some embodiments, the subject has or is suspected of having a condition associated with proliferative disorder or a microbial infection.

In some embodiments, the disclosed composition is formulated to be compatible with its intended route of administration. For example, the nucleic acid constructs, recombinant cells, recombinant polypeptides, and/or pharmaceutical compositions of the disclosure can be given orally or by inhalation, but it is more likely that they will be administered through a parenteral route. Examples of parenteral routes of administration include, for example, intravenous, intranodal, intradermal, subcutaneous, transdermal (topical), transmucosal, intravaginal, intraocular, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject nucleic acid constructs, recombinant cells, recombinant polypeptides, and/or pharmaceutical compositions of the disclosure can be determined by standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally suitable. While compounds that exhibit toxic side effects can be used, care should be taken to design a delivery system that targets such compounds to the site of affected tissue in order to minimize potential damage to uninfected cells and, thereby, reduce side effects.

For example, the data obtained from the cell culture assays and animal studies can be used in formulating a range of dosage for use in humans. The dosage of such compounds lies generally within a range of circulating concentrations that include the ED₅₀ with little or no toxicity. The dosage can vary within this range depending upon the dosage form employed and the route of administration utilized. For any compound used in the method of the disclosure, the therapeutically effective dose can be estimated initially from cell culture assays. A dose can be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma can be measured, for example, by high performance liquid chromatography.

The therapeutic compositions described herein, e.g., nucleic acid constructs, recombinant cells, recombinant polypeptides, and/or pharmaceutical compositions, can be administered one from one or more times per day to one or more times per week; including once every other day. The skilled artisan will appreciate that certain factors can influence the dosage and timing required to effectively treat a subject, including but not limited to the severity of the disease, previous treatments, the general health and/or age of the subject, and other diseases present. Moreover, treatment of a subject with a therapeutically effective amount of the subject multivalent polypeptides and multivalent antibodies of the disclosure can include a single treatment or, can include a series of treatments. In some embodiments, the compositions are administered every 8 hours for five days, followed by a rest period of 2 to 14 days, e.g., 9 days, followed by an additional five days of administration every 8 hours. With regard to nucleic acid constructs and recombinant polypeptides, the therapeutically effective amount of a nucleic acid construct or recombinant polypeptide of the disclosure (e.g., an effective dosage) depends on the nucleic acid construct or recombinant polypeptide selected. For instance, single dose amounts in the range of approximately 0.001 to 0.1 mg/kg of patient body weight can be administered. In some embodiments, about 0.005, 0.01, 0.05 mg/kg can be administered. In some embodiments, single dose amounts in the range of approximately 0.03 μg to 300 μg/kg of patient body weight can be administered. In some embodiments, single dose amounts in the range of approximately 0.3 mg to 3 mg/kg of patient body weight can be administered.

As discussed supra, a therapeutically effective amount includes an amount of a therapeutic composition that is sufficient to promote a particular effect when administered to a subject, such as one who has, is suspected of having, or is at risk for a health condition, e.g., a disease or infection. In some embodiments, an effective amount includes an amount sufficient to prevent or delay the development of a symptom of the disease or infection, alter the course of a symptom of the disease or infection (for example but not limited to, slow the progression of a symptom of the disease or infection), or reverse a symptom of the disease or infection. It is understood that for any given case, an appropriate effective amount can be determined by one of ordinary skill in the art using routine experimentation.

The efficacy of a treatment including a disclosed therapeutic composition for the treatment of disease or infection can be determined by the skilled clinician. However, a treatment is considered effective treatment if at least any one or all of the signs or symptoms of disease or infection are improved or ameliorated. Efficacy can also be measured by failure of an individual to worsen as assessed by hospitalization or need for medical interventions (e.g., progression of the disease or infection is halted or at least slowed). Methods of measuring these indicators are known to those of skill in the art and/or described herein. Treatment includes any treatment of a disease or infection in a subject or an animal (some non-limiting examples include a human, or a mammal) and includes: (1) inhibiting the disease or infection, e.g., arresting, or slowing the progression of symptoms; or (2) relieving the disease or infection, e.g., causing regression of symptoms; and (3) preventing or reducing the likelihood of the development of symptoms.

In some embodiments, the nucleic acid constructs, recombinant cells, recombinant polypeptides, and/or pharmaceutical compositions of the disclosure can be administered to a subject in a composition having a pharmaceutically acceptable carrier and in an amount effective to stimulate an immune response. Generally, a subject can be immunized through an initial series of injections (or administration through one of the other routes described below) and subsequently given boosters to increase the protection afforded by the original series of administrations. The initial series of injections and the subsequent boosters are administered in such doses and over such a period of time as is necessary to stimulate an immune response in a subject. In some embodiments, the administered composition results in an increased production of interferon in the subject. In some embodiments of the disclosed methods, the subject is a mammal. In some embodiments, the mammal is human.

As described above, pharmaceutically acceptable carriers suitable for injectable use include sterile aqueous solutions (where water soluble) or dispersions and sterile powders for the extemporaneous preparation of sterile injectable solutions or dispersions. In these cases, the composition must be sterile and must be fluid to the extent that easy syringability exists. The composition must further be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, etc.), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like.

Sterile injectable solutions can be prepared by incorporating the nucleic acid constructs, recombinant cells, and/or recombinant polypeptides in the required mount in an appropriate solvent with one or a combination of ingredients enumerated above, as required, followed by filtered sterilization.

When the nucleic acid constructs, recombinant cells, recombinant polypeptides, and/or pharmaceutical compositions are suitably protected, as described above, they can be orally administered, for example, with an inert diluent or an assimilable edible carrier. The nucleic acid constructs, recombinant cells, recombinant polypeptides, and/or pharmaceutical compositions and other ingredients can also be enclosed in a hard or soft shell gelatin capsule, compressed into tablets, or incorporated directly into the individual's diet. For oral therapeutic administration, the active compound can be incorporated with excipients and used in the form of ingestible tablets, buccal tablets, troches, capsules, elixirs, suspensions, syrups, wafers, and the like.

In some embodiments, the nucleic acid constructs and recombinant polypeptides of the disclosure can be delivered to a cell or a subject by a lipid-based nanoparticle (LNP). LNP are generally less immunogenic than viral particles. While many humans have preexisting immunity to viral particles there is no pre-existing immunity to LNP. In addition, adaptive immune response against LNP is unlikely to occur which enables repeat dosing of LNP.

Several different ionizable cationic lipids have been developed for use in LNP. These include C12-200, MC3, LN16, and MD1 among others. For example, in one type of LNP, a GalNAc moiety is attached to the outside of the LNP and acts as a ligand for uptake in to the liver via the asialyloglycoprotein receptor. Any of these cationic lipids can be used to formulate LNP for delivery of the nucleic acid constructs and recombinant polypeptides of the disclosure to the liver.

In some embodiments, a LNP refers to any particle having a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm.

LNPs can be made from cationic, anionic, or neutral lipids. Neutral lipids, such as the fusogenic phospholipid DOPE or the membrane component cholesterol, can be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses. LNPs can also have hydrophobic lipids, hydrophilic lipids, or both hydrophobic and hydrophilic lipids.

Any lipid or combination of lipids that are known in the art can be used to produce a LNP. Examples of lipids used to produce LNPs are: DOTMA, DOSPA, DOTAP, DMRIE, DC-cholesterol, DOTAP—cholesterol, GAP-DMORIE—DPyPE, and GL67A—DOPE—DMPE—polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerC14, and PEG-CerC20.

In some embodiments, the lipids can be combined in any number of molar ratios to produce a LNP. In addition, the polynucleotide(s) can be combined with lipid(s) in a wide range of molar ratios to produce a LNP.

In some embodiments, the therapeutic compositions described herein, e.g., nucleic acid constructs, recombinant cells, recombinant polypeptides, and/or pharmaceutical compositions are incorporated into therapeutic compositions for use in methods of preventing or treating a subject who has, who is suspected of having, or who may be at high risk for developing a cancer, an autoimmune disease, and/or an infection.

In some embodiments, the therapeutic compositions described herein, e.g., nucleic acid constructs, recombinant cells, recombinant polypeptides, and/or pharmaceutical compositions are incorporated into therapeutic compositions for use in methods of preventing or treating a subject who has, who is suspected of having, or who may be at high risk for developing a microbial infection. In some embodiments, the microbial infection is a bacterial infection. In some embodiments, the microbial infection is a fungal infection. In some embodiments, the microbial infection is a viral infection.

### Additional Therapies

In some embodiments, a composition according to the present disclosure is administered to the subject individually as a single therapy (monotherapy) or as a first therapy in combination with at least one additional therapies (e.g., second therapy). In some embodiments, the second therapy is selected from the group consisting of chemotherapy, radiotherapy, immunotherapy, hormonal therapy, toxin therapy, targeted therapy, and surgery. In some embodiments, the second therapy is selected from the group consisting of chemotherapy, radiotherapy, immunotherapy, hormonal therapy, toxin therapy or surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Kits

Also provided herein are various kits for the practice of a method described herein. In particular, some embodiments of the disclosure provide kits for eliciting an immune response in a subject. Some other embodiments relate to kits for the prevention of a health condition in a subject in need thereof. Some other embodiments relate to kits for methods of treating a health condition in a subject in need thereof. For example, provided herein, in some embodiments, are kits that include one or more of the nucleic acid constructs, recombinant cells, recombinant polypeptides, and/or pharmaceutical compositions as provided and described herein, as well as written instructions for making and using the same.

In some embodiments, the kits of the disclosure further include one or more means useful for the administration of any one of the provided nucleic acid constructs, recombinant cells, recombinant polypeptides, and/or pharmaceutical compositions to a subject. For example, in some embodiments, the kits of the disclosure further include one or more syringes (including pre-filled syringes) and/or catheters (including pre-filled syringes) used to administer any one of the provided nucleic acid constructs, recombinant cells, recombinant polypeptides, and/or pharmaceutical compositions to a subject. In some embodiments, a kit can have one or more additional therapeutic agents that can be administered simultaneously or sequentially with the other kit components for a desired purpose, e.g., for diagnosing, preventing, or treating a condition in a subject in need thereof.

Any of the above-described kits can further include one or more additional reagents, where such additional reagents can be selected from: dilution buffers; reconstitution solutions, wash buffers, control reagents, control expression vectors, negative controls, positive controls, reagents suitable for in vitro production of the provided nucleic acid constructs, recombinant cells, recombinant polypeptides, and/or pharmaceutical compositions of the disclosure.

In some embodiments, the components of a kit can be in separate containers. In some other embodiments, the components of a kit can be combined in a single container.

In some embodiments, a kit can further include instructions for using the components of the kit to practice the methods disclosed herein. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, N.Y.: Cold Spring Harbor Laboratory and Sambrook, J., & Russel, D. W. (2001). Molecular Cloning: A Laboratory Manual (3rd ed.). Cold Spring Harbor, N.Y.: Cold Spring Harbor Laboratory (jointly referred to herein as “Sambrook”); Ausubel, F. M. (1987). Current Protocols in Molecular Biology. New York, N.Y.: Wiley (including supplements through 2014); Bollag, D. M. et al. (1996). Protein Methods. New York, N.Y.: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, Calif.: Academic Press; Lefkovits, I. (1997). The Immunology Methods Manual: The Comprehensive Sourcebook of Techniques. San Diego, Calif.: Academic Press; Doyle, A. et al. (1998). Cell and Tissue Culture: Laboratory Procedures in Biotechnology. New York, N.Y.: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, N.Y.: Cold Spring Harbor Laboratory Press; Beaucage, S. L. et al. (2000). Current Protocols in Nucleic Acid Chemistry. New York, N.Y.: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1. Construction of CHIKV Vectors

This Example describes the results of experiments performed to construct a number of base CHIKV vectors (e.g., without a heterologous gene) that were subsequently used for expression of a gene of interest, e.g., (i) hemagglutinin precursor (HA) of the influenza A virus H5N1, or (ii) a synthetic sequence cassette encoding genes or parts of genes relevant to oncology such as estrogen receptor alpha (ESR1), epidermal growth factor receptor 2 (HER2), and human epidermal growth factor receptor (HER3), or reporter genes such as red firefly luciferase, or cytokines such as interleukin-1 receptor antagonist (IL-1RA) or interleukin-12 (IL12). Alternatively, a human papillomavirus (HPV) E6/E7 gene can also be used.

An initial observation was made that the publicly available alphavirus genomic data does not always provide nucleotide sequences that are capable of direct replacement of the nuclei acid sequences encoding the structural proteins with a gene of interest (GOI) to result in self-replicating RNA and transgene-expressing replicons. As described in greater detail below, while it was possible to replace the structural polyprotein gene in CHIKV strain S27 (Genbank AF369024) with a synthetic HPV E6/E7 gene (human papillomavirus E6/E7 gene) (see, e.g., FIG. 2C), a hemagglutinin (HA) gene from the influenza A virus H5N1 (see, e.g., FIG. 3B), or a red firefly luciferase gene can also be used to produce a replicon capable of RNA replication and transgene expression in transfected BHK-21 cells, the same synthetic sequence used to similarly replace the structural polyprotein gene in CHIKV strain DRDE-06 (Genbank EF210157) was unable to undergo RNA replication or express the transgene. Thus, simple replacement of CHIKV structural proteins with heterologous genes using available, published sequences would not be insufficient for generation of functional replicons. Stated differently, further engineering, such as using heterologous 5′ and/or 3′ UTR sequences, would be required to create replicon systems suitable for use in vaccines and therapeutics.

As described in greater detail below, despite the numerous evolutionary divergences in sequence found across the genomes of CHIKV strains S27 and DRDE-06, the experimental data presented herein has demonstrated that a functional CHIKV strain DRDE replicon could be generated by replacing the DRDE 3′ UTR with the 3′ UTR from CHIKV strain S27 (see, e.g., FIG. 2C).

In these experiments, the base CHIKV vectors (S27 and DRDE-06) were synthesized de novo in four ˜4 kb parts (Twist Bioscience, Thermo Fisher GeneArt) from reference sequences (Genbank AF369024 and EF210157 for strains S27 and DRDE-06, respectively) with a unique restriction enzyme cut site (SpeI, 5′-A′CTAG,T-3′) in place of the coding sequence of the CHIKV structural genes (where the 5′ A matches the location of the structural polyprotein's ATG start codon, and the 3′ T matches the location of the structural polyprotein's stop codon TAA). A bacteriophage T7 RNA polymerase promoter (5′-TAATACGACTCACTATAG-3′; SEQ ID NO: 7) was included upstream of the CHIKV genome sequence, and downstream contained a (37-A) polyA sequence followed by a T7 terminator sequence (5′-AACCCCTCTCTAAACGGAGGGGTTTTTTT-3′; SEQ ID NO: 8) followed by a unique restriction enzyme cut site (NotI, 5′-GC′GGCC,GC-3′). The parts were combined in a five-piece Gibson Assembly® reaction a linearized pYL backbone and the four synthesized fragments to result in the CHIKV base vectors. The resulting CHIKV S27 and CHIKV DRDE base vectors are SEQ ID NO: 1 and SEQ ID NO: 2, respectively. SEQ ID NO: 1 corresponds to the CHIKV S27 base vector containing S27 5′ UTR and S27 3′ UTR sequences. SEQ ID NO: 2 corresponds to the CHIKV DRDE base vector containing DRDE 5′ UTR and DRDE 3′ UTR sequences. It was observed that this CHIKV DRDE base vector is unable to undergo replication.

A modified CHIKV DRDE base vector was constructed as follows. The CHIKV DRDE vector (FIG. 3C) was constructed by SpeI and NotI restriction digestion of the DRDE-06 base vector and was combined in a two-piece Gibson Assembly® reaction with the linearized backbone, and a PCR product from the CHIKV S27 vector containing the 3′ UTR (SEQ ID NO: 6), polyA, and T7 terminator sequence (SEQ ID NO: 8). The resulting CHIKV DRDE base vector is SEQ ID NO: 3. This CHIKV DRDE base vector contained DRDE 5′ UTR and S27 3′ UTR sequences, and was found to be able to undergo replication.

CHIKV vectors encoding an expression reporter gene were constructed as follows. A red firefly luciferase (rFF) reporter gene was synthesized and inserted into the CHIKV base vectors described above by SpeI restriction endonuclease digestion and two-fragment Gibson Assembly® reaction with a PCR product containing the rFF gene with homologous ends to the linearized base vectors.

The CHIKV S27 vector (FIG. 2B) was constructed by SpeI restriction digestion of the S27 base vector and was combined in a two-piece Gibson Assembly® reaction with the linearized backbone and a PCR product containing the synthetic HPV E6/E7 gene.

The CHIKV DRDE vector (FIG. 2C) was constructed by SpeI and NotI restriction digestion of the DRDE base vector and was combined in a three-piece Gibson Assembly® reaction with (i) the linearized backbone, (ii) a PCR product containing the synthetic HPV E6/E7 gene, and (iii) a PCR product from the CHIKV S27 vector containing the 3′ UTR (SEQ ID NO: 6), polyA, and T7 terminator sequence (SEQ ID NO: 8).

CHIKV vectors expressing HA were constructed as follows. The hemagglutinin (HA) gene from influenza (Genbank AY651334) was codon optimized/refactored for human expression in silico and synthesized de novo (IDT) and inserted into the CHIKV base vectors described above by SpeI restriction endonuclease digestion and two-fragment Gibson Assembly® reaction with a PCR product containing the HA gene with homologous ends to the linearized base vectors. The resulting CHIKV S27 and CHIKV DRDE vectors are described in FIG. 3B and FIG. 3C respectively.

CHIKV vectors encoding an expression cassette for human IL-1RA and IL-12 were similarly constructed, by insertion of the synthetic cassette into the CHIKV base vectors described above by SpeI restriction endonuclease digestion and two-fragment Gibson Assembly® reaction with a PCR product containing the synthetic cassette with homologous ends to the linearized base vectors.

CHIKV vectors expressing oncology-related polypeptides were similarly constructed by a two-fragment Gibson Assembly® procedure (Gibson et al., Nat. Methods 6, 343-345, 2009) of a PCR product containing sequences from ESR1, HER2, and HER3 with homologous ends to the SpeI-linearized base CHIKV vectors. The resulting CHIKV S27 and CHIKV DRDE vectors are described in FIGS. 3F and 3G respectively.

The control VEE vectors described in FIG. 2A, FIG. 3A, FIG. 3E, the rFF reporter, and the human IL-1RA and IL-12 cassette were constructed similarly as described above for the CHIKV base vectors. The control VEE vector was synthesized de novo from a reference sequence (Genbank L01443).

### Example 2. Construction of SINV Vectors

This Example describes the design and construction of a number of base SINV vectors (e.g., without a heterologous gene) that were subsequently used for expression of a gene of interest (e.g., hemagglutinin precursor (HA) of the influenza A virus H5N1, or a synthetic sequence cassette encoding genes or parts of genes relevant to oncology such as ESR1, HER2, and HER3, or red firefly luciferase, or cytokines such as IL-1RA or IL12). Alternatively, a human papillomavirus (HPV) E6/E7 gene can also be used.

The base SINV Girdwood vector was synthesized de novo in four ˜4 kb parts (Twist Bioscience, Thermo Fisher GeneArt) from a Girdwood strain reference sequence (Genbank MF459683) with a unique restriction enzyme cut site (SpeI, 5′-A′CTAG,T-3′) in place of the coding sequence of the SINV structural genes (where the 5′ A is the next nucleotide after a P2A sequence following nucleotide 93 of the structural polyprotein gene, and the 3′ T matches the location of the structural polyprotein's stop codon TGA). A bacteriophage T7 RNA polymerase promoter (5′-TAATACGACTCACTATAG-3′; SEQ ID NO: 7) was included upstream of the SINV genome sequence, and downstream contained a (37-A) polyA sequence followed by a T7 terminator sequence (5′-AACCCCTCTCTAAACGGAGGGGTTTTTTT-3′; SEQ ID NO: 8) followed by a unique restriction enzyme cut site (NotI, 5′-GC′GGCC,GC-3′). The parts were combined in a five-piece Gibson Assembly® reaction (e.g., a linearized pYL backbone and the four synthesized fragments) to result in the SINV Girdwood base vector (SEQ ID NO: 4).

The base SINV AR86 vector was synthesized de novo in four ˜4 kb parts (Twist Bioscience) from an AR86 reference sequence (Genbank U38305) with a unique restriction enzyme cut site (SpeI, 5′-A′CTAG,T-3′) in place of the coding sequence of the SINV structural genes (where the 5′ A is the next nucleotide after a P2A sequence following nucleotide 93 of the structural polyprotein gene, and the 3′ T matches the location of the structural polyprotein's stop codon TGA). A bacteriophage T7 RNA polymerase promoter (5′-TAATACGACTCACTATAG-3′; SEQ ID NO: 7) was included upstream of the SINV genome sequence, and downstream contained a (37-A) polyA sequence followed by a T7 terminator sequence (5′-AACCCCTCTCTAAACGGAGGGGTTTTTTT-3′; SEQ ID NO: 8) followed by a unique restriction enzyme cut site (NotI, 5′-GC′GGCC,GC-3′). The parts were combined in a five-piece Gibson Assembly® reaction (e.g., a linearized pYL backbone and the four synthesized fragments) to result in the SINV AR86 base vector, which also contained additional modifications to improve functionality.

SINV vectors encoding an expression reporter gene were constructed as follows. A red firefly luciferase (rFF) reporter gene was synthesized and inserted into the SINV base vectors described above by SpeI restriction endonuclease digestion and two-fragment Gibson Assembly® reaction with a PCR product containing the rFF gene with homologous ends to the linearized base vectors.

The SINV vector described in FIG. 3D was constructed as follows. The hemagglutinin (HA) gene from influenza (Genbank AY651334) was codon optimized/refactored for human expression in silico and synthesized de novo (IDT) and inserted into the SINV Girdwood base vector described above by SpeI restriction digest and two-fragment Gibson Assembly® reaction with a PCR product containing the HA gene with homologous ends to the base vector to result in the final vector. In another experiment, a SINV AR86 vector containing a codon-optimized HA gene was similarly constructed using the SINV AR86 base vector described in Example 2 above (data not shown).

SINV vectors encoding an expression cassette for human IL-1RA and IL-12 were similarly constructed, by insertion of the synthetic cassette into the SINV base vectors described above by SpeI restriction endonuclease digestion and two-fragment Gibson Assembly® reaction with a PCR product containing the synthetic cassette with homologous ends to the linearized base vectors.

SINV vectors expressing oncology-related polypeptides were similarly constructed by two fragment Gibson Assembly® reaction of a PCR product containing sequences from ESR1, HER2, and HER3 with homologous ends to the SpeI-linearized base SINV vectors. A schematic representation of the SINV Girdwood vector is described in FIG. 3I.

### Example 3. In Vitro Evaluation of Modified CHIKV and SINV Vectors

This Example describes the results of in vitro experiments performed to evaluate expression levels of the synthetic CHIKV and SINV replicon constructs described in Examples 1 and 2 above, and to investigate any differential behavior thereof (e.g., replication and protein expression).

In these experiments, synthetic replicon constructs derived from the following alphaviruses were designed and subsequently evaluated: VEE, CHIKV S27, CHIKV DRDE, SINV AR86, and SINV Girdwood.

In vitro transcription: RNA was prepared by in vitro transcription using bacteriophage T7 polymerase with either a 5′ ARCA cap (HiScribe™ T7 ARCA mRNA Kit, NEB) or by uncapped transcription (HiScribe™ T7 High Yield RNA Synthesis Kit, NEB) followed by addition of a 5′ cap 1 (Vaccinia Capping System, mRNA Cap 2′-O-Methyltransferase, NEB). RNA was then purified using phenol/chloroform extraction, or column purification (Monarch® RNA Cleanup Kit, NEB). RNA concentration was determined by absorbance at 260 nm (Nanodrop, Thermo Fisher Scientific).

Replication: RNA was transformed by electroporation into BHK-21 or Vero cells (e.g., 4D-Nucleofector™, Lonza). At 18-20 hours following transformation, the cells were fixed and permeabilized (eBioscience™ Foxp3/Transcription Factor Staining Buffer Set, Invitrogen) and subsequently stained using a PE-conjugated anti-dsRNA mouse monoclonal antibody (J2, Scicons) to quantify the frequency of dsRNA+ cells and the mean fluorescence intensity (MFI) of dsRNA in individual cells by fluorescence flow cytometry.

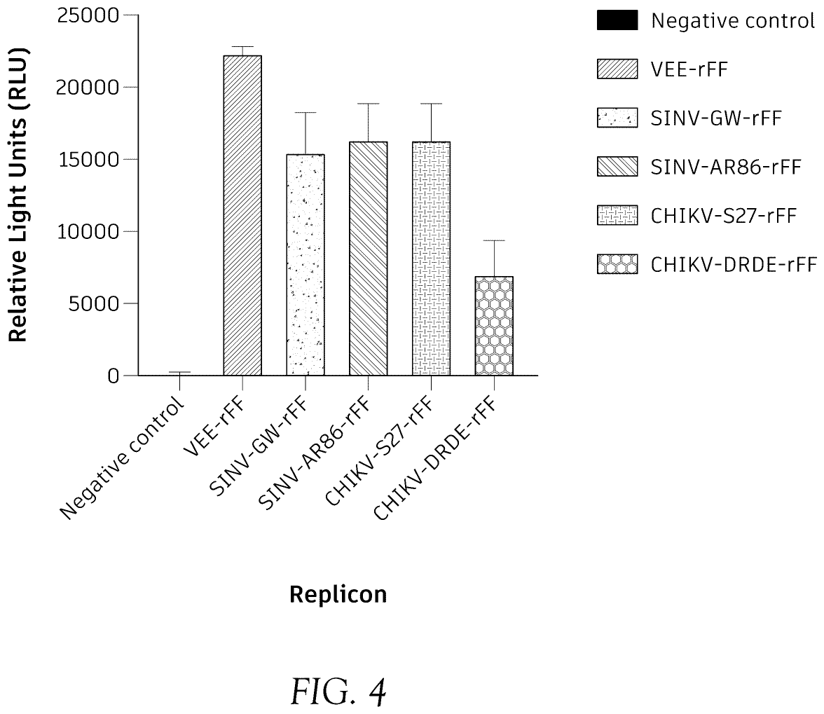

Protein expression: RNA was transformed by electroporation into BHK-21 or Vero cells (e.g., 4D-Nucleofector™, Lonza). At 18-20 hours following transformation, the expression of transgene(s) were quantified. For replicons expressing rFF, luciferase activity was quantified using the Luciferase Assay System protocol (Promega) (FIG. 4). The detection of luminescence from these replicon-transfected cells demonstrates that the replicons were able to undergo RNA replication and express a gene of interest (GOI). In this example the GOI is a reporter enzyme that exhibits biological function. In alternative experiments, the cells can be fixed and permeabilized (eBioscience™ Foxp3/Transcription Factor Staining Buffer Set, Invitrogen) and stained using a FITC-conjugated anti-LAMP1 mouse monoclonal antibody (HA3, BioLegend) to quantify the frequency of LAMP-fusion protein+ cells and the mean fluorescence intensity (MFI) of the LAMP-fusion protein in individual cells by fluorescence flow cytometry.

For replicons expressing IL-12, media was collected from cells and IL-12 was quantified by ELISA (R&D Biosystems) or in an IL-12 bioactivity assay (Promega). For replicons expressing IL-1RA, media was collected and IL-1RA was quantified by ELISA (R&D Biosystems) or in a bioactivity assay using HEK-Blue™ IL-1R cells (InvivoGen) as described. HEK-Blue™ IL-1R cells express SEAP in response to IL-1B. First, 5E4 HEK-Blue™ IL-1R cells were seeded per well on 96-well plates. The HEK-Blue™ IL-1R cells were incubated with serial dilutions of test media in duplicate wells for 40 minutes. To generate a standard curve, the HEK-Blue™ IL-1R cells were incubated with a titrated concentration of recombinant IL-1RA (Peprotech) in duplicate wells for 40 minutes. Recombinant IL-1B (InvivoGen) was then added to each well to a final concentration of 1 ng/ml. The following day, SEAP expression was quantified using QUANTI-Blue™ Solution (InvivoGen) to quantify Il-1RA bioactivity.

Additional experiments: BHK-21 or Vero cells are pre-treated with a recombinant interferon (IFN) prior to electroporation of RNA, and impacts on replication and protein expression for each vector are measured using the assays described above.

### Example 4. In Vivo Evaluation of Modified CHIKV and SINV Vectors—Influenza

This Example describes the results of in vivo experiments performed to evaluate any differential immune responses following vaccination with the synthetic CHIKV and SINV replicon constructs described in Examples 1 and 2 above (e.g., both unformulated and LNP formulated vectors).

In these experiments, synthetic replicon constructs derived from the following alphaviruses were designed and subsequently evaluated: VEE, CHIKV S27, CHIKV DRDE, SINV AR86, and SINV Girdwood.

Mice and injections: Female BALB/c mice were purchased from Charles River Labs, Envigo, or Jackson Laboratories. On day of dosing, 10 μg (single dose groups, i.e. prime only) or 5 μg (two dose groups, i.e. prime-boost) of material was injected intramuscularly split into both quadricep muscles. Vectors were administered either unformulated in saline, or LNP-formulated. Animals were monitored for body weight and other general observations throughout the course of the study. For immunogenicity studies, animals were dosed on Day 0 and Day 21 (two dose groups only). Spleens was collected at Day 14 (single 10 μg dose groups) and at Day 35 (single 5 μg dose groups), and serum was isolated at Days 14 and 35. For protein expression studies, animals can be dosed on Day 0, and bioluminescence can be assessed on Days 1, 3, and 7. In vivo imaging of luciferase activity can be done using an IVIS system at the indicated time points.

LNP formulation: Replicon RNA was formulated in lipid nanoparticles using a microfluidics mixer and analyzed for particle size, polydispersity using dynamic light scattering and encapsulation efficiency. In this Example, molar ratios of lipids used in formulating LNP particles was 35% C12-200, 46.5% Cholesterol, 2.5% PEG-2K and 16% DOPE.

ELISpot: For detection of T cell responses following immunization, mouse IFNγ ELISpot kit (Mabtech) was used as per the manufacturer's protocol. In brief, single splenocyte suspensions were prepared and plated at 5×10⁶ cells/ml in AIM V media with the following stimulation conditions: media only (mock), PMA and ionomycin (positive control) and CD4 (KSSFFRNVVWLIKKN) (SEQ ID NO: 9) and CD8 (IYSTVASSL) (SEQ ID NO: 10) peptides from HA from Influenza A/Vietnam/2004/1203 (H5N1). Peptides were used at 1-10 μg/ml final concentration. Spot-forming units were imaged and quantified and plotted per million cells.

The magnitude of HPV-specific T cell responses can be measured as following: IFNγ ELISpot analysis can be performed using Mouse IFNγ ELISpot PLUS Kit (HRP) (MabTech) as per manufacturer's instructions. In brief, splenocytes can be isolated and resuspended to a concentration of 5×10⁶ cells/mL in media containing peptides representing either CD4+ or CD8+ T cell epitopes to HPV, PMA/ionomycin as a positive control, or DMSO as a mock stimulation.

Intracellular cytokine staining. Spleens can be isolated according to the methods outlined for ELISpots, and 1×10⁶ cells can be added to cells containing media in a total volume of 200 μL per well. Each well can contain peptides representing either CD4+ or CD8+ T cell epitopes to HPV, PMA/ionomycin as a positive control, or DMSO as a mock stimulation. After 1 hour, GolgiPlug™ protein transport inhibitor (BD Biosciences) can be added to each well. Cells can be incubated for another 5 hours. Following incubation, cells can be surface stained for CD8+(53-6.7), CD4+(GK1.5), B220 (B238128), Gr-1 (RB6-8C5), CD16/32 (M93) using standard methods. Following surface staining, cells can be fixed and stained for intracellular proteins as per standard methods for IFNγ (RPA-T8), IL-2 (JES6-5H4), and TNF (MP6-XT22). Cells can then be subsequently analyzed on a flow cytometer and the acquired FCS files analyzed using FlowJo software version 10.4.1.

Antibodies. Antibody responses to measure total HPV E6/E7-specific IgG can be measured using ELISA kits from Alpha Diagnostic International as per manufacturer's instructions.

Hemagglutination Inhibition Assay (HAI): To prepare serum, RDE solution was prepared according to the manufacturer instructions. Unused solution can be stored in 1 mL aliquots at −15° to −25° C. for up to one year. 20 μL serum was pipetted for each sample to be tested and a positive control in separate microcentrifuge tubes. 80 μL RDE was added to each tube. Amounts can be increased to using the same ratio (e.g., 40 μL serum and 160 μL RDE) if additional serum is needed. High titer sera and positive controls can be pre-diluted prior to adding RDE (e.g., 5 μL serum and 20 μL PBS, plus 100 μL RDE). The samples and the positive control were incubated in a 37° C.±1° C. water bath for 18-20 hours (Day 1). The sera and RDE were inactivated by heating in a 56° C.±2° C. water bath for 35-45 minutes (Day 2). The sera were centrifuged briefly to remove condensation from lids of tubes. 100 μL 1×PBS was added for each 100 μL treated sera for a starting dilution of 1:10. Virus was diluted to a 4 hemagglutinating units/25 μL in 1×PBS/0.75% BSA and placed on wet ice. For the control plate, 25 μL PBS was added to column 9-10 of a 96 well V-bottom plate. 50 μL OBS was added to columns 11-12, but only rows C-H. For sample plates, 25 μL PBS was added to all wells in row B-H (column 1-12). 50 μL serum samples were added to duplicate adjacent wells in row A. 25 μL negative control serum was added to wells 11A, 11B, 12A, 12B on the control plate. 25 μL positive control was added to wells 9A and 10A on the control plate. On the sample plate(s), row A (columns 1-12) was mixed 3-4 times. 25 μL was transferred to row B, continuing two-fold dilutions to row H. 25 μL from row H was discarded. On the control plate, row A (columns 1-10) was mixed 3-4 times. 25 μL was transferred to row B, continuing two-fold dilutions to row H. 25 μL from row H was discarded. 25 μL diluted virus was added to all wells of the sample plate(s) and to columns 9-10 of the control plate. Virus was also added to wells 11A, 11B, 12A, and 12B on the control plate. 50 μL diluted virus was added to wells 11E and 12E, mixed 3-4 times, and two-fold dilutions were performed to wells 11H and 12H. Plates were incubated at room temperature for 50-60 minutes. 50 μL 1.1% HRBCs was added to all wells. Plates were incubated at room temperature for 50-60 minutes. The plates were tilted to read agglutination pattern.

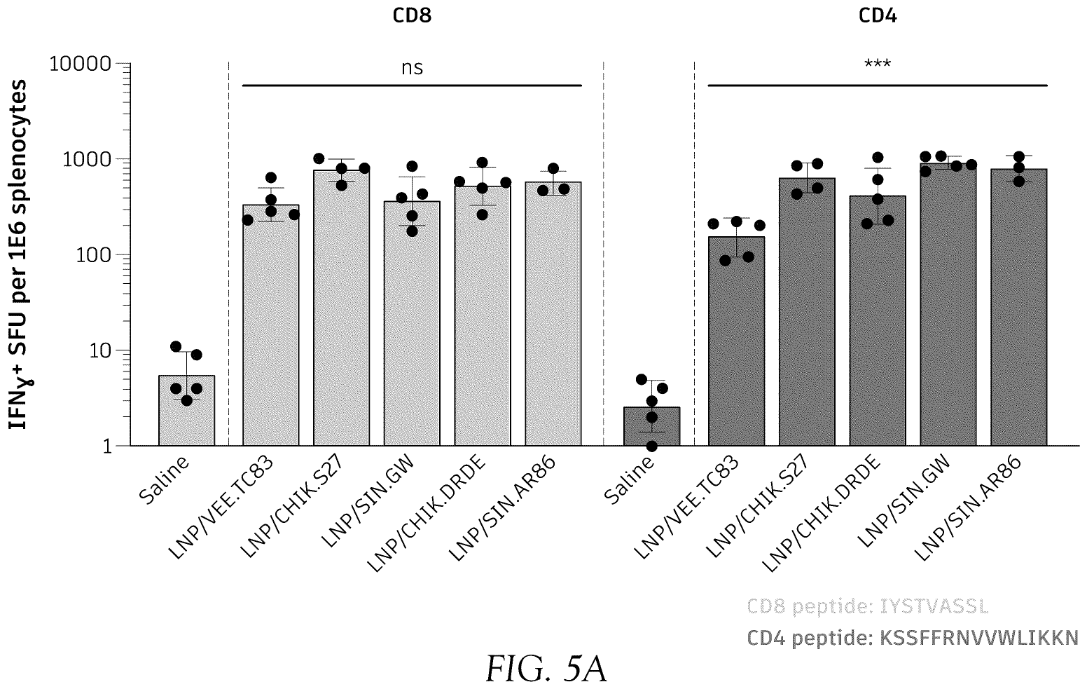

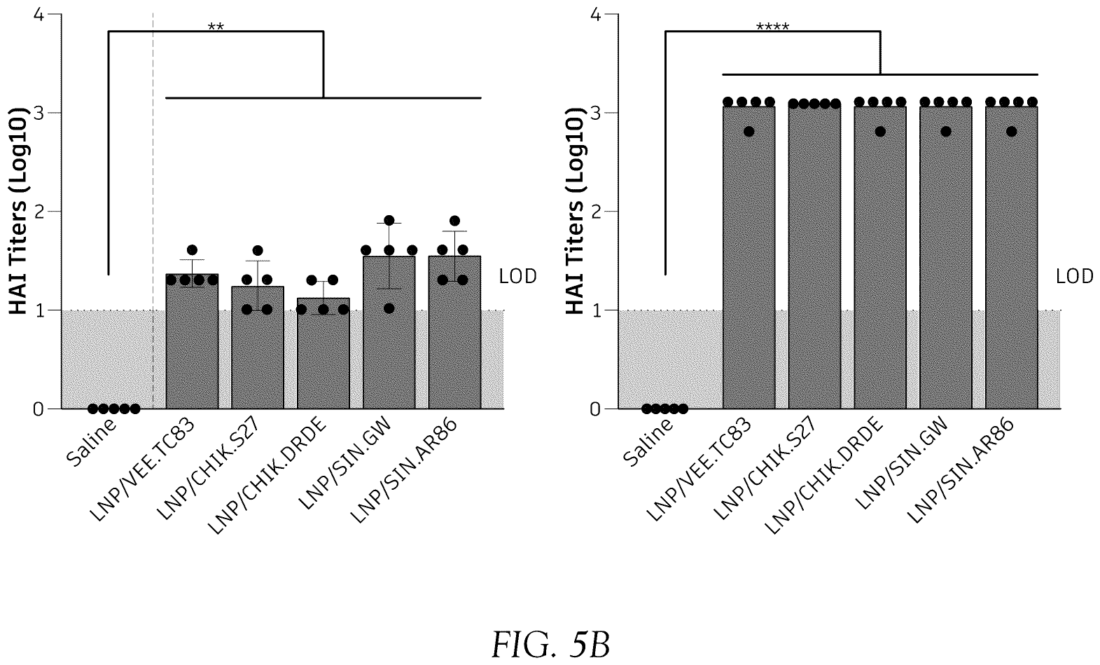

To assess the in vivo impact of CHIKV- and SINV-derived vectors, in this experiment, they were compared to a VEE synthetic replicon derived from TC-83, which is commonly used within the field. In this example, the LNP-formulated material demonstrates HAI titers 14 days after a single dose in all of the animals across each vector. Similarly, all LNP-formulated vectors generated both CD4+ and CD8+ HA-specific T cell responses in the spleen as measured by ELISpot analysis. Here, it was observed that some of the CHIKV- and SINV-derived vectors have significantly improved CD4+ or CD8+ T cell responses depending on which epitope is being used for stimulation (FIGS. 5A-5B). This demonstrated that the CHIKV- and SINV-derived vectors themselves could generate differential responses compared with stereotypic VEE-based vectors. Higher T cell and antibody responses against the encoded protein would be desirable for use as vaccines, whereas lower T cell and antibody responses against the encoded protein would be preferable for biotherapeutics.

### Example 5. In Vivo Evaluation of Modified CHIKV and SINV Vectors—Oncology

This Example describes the results of in vivo experiments performed to evaluate any differential immune responses following vaccination with the synthetic CHIKV and SINV replicon constructs described in Examples 1 and 2 above (e.g., both unformulated and LNP formulated vectors). In these experiments, synthetic replicon constructs derived from the following alphaviruses were designed and subsequently evaluated: VEE, CHIKV S27, CHIKV DRDE, SINV AR86, and SINV Girdwood.

Mice and injections. Female BALB/c mice were purchased from Charles River Labs, Envigo, or Jackson Laboratories. On day of dosing, 10 μg of material was injected intramuscularly split into both quadricep muscles. Vectors were administered either unformulated in saline, or LNP-formulated. Animals were monitored for body weight and other general observations throughout the course of the study. For immunogenicity studies, animals were dosed on Day 0 and Day 21. Spleens were collected at Day 35.

LNP formulation. Replicon RNA was formulated in lipid nanoparticles using a microfluidics mixer and analyzed for particle size, polydispersity using dynamic light scattering and encapsulation efficiency. Molar ratios of lipids used in formulating LNP particles was 35% C12-200, 46.5% Cholesterol, 2.5% PEG-2K and 16% DOPE. Alternatively, a ready-to-use formulation of lipids obtained from another entity, such as those provided by Precision Nanosystems, Inc. can be used.

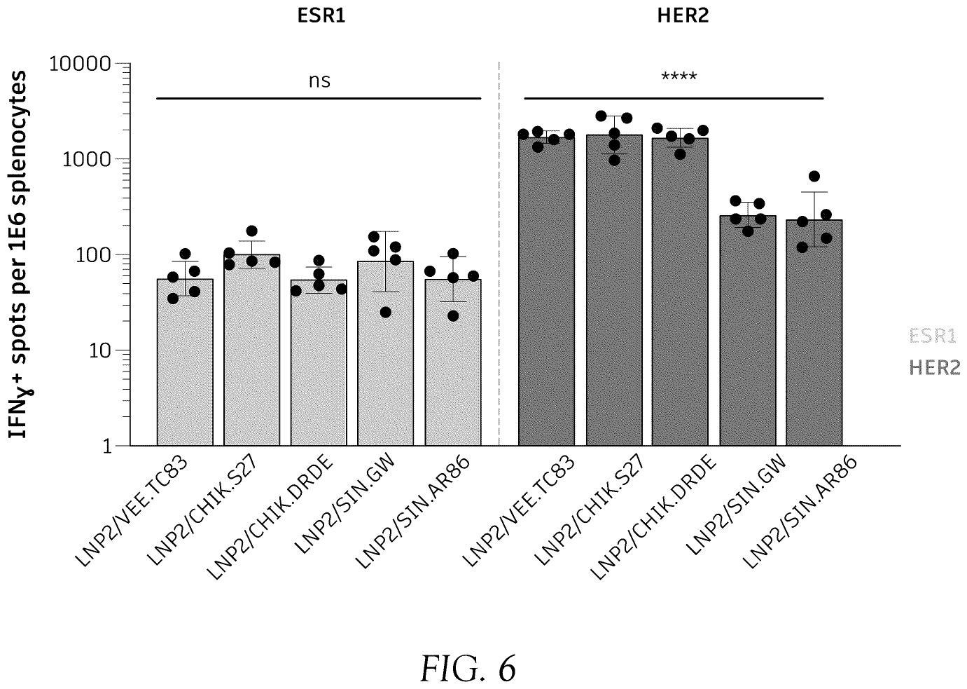

ELISpot. To measure the magnitude of ESR1-, HER2-, or HER3-specific T cell responses, IFNγ ELISpot analysis was performed using Mouse IFNγ ELISpot PLUS Kit (HRP) (MabTech) as per manufacturer's instructions. In brief, splenocytes were isolated and resuspended to a concentration of 5×10⁶ cells/mL in media containing peptides representing either CD4+ or CD8+ T cell epitopes to ESR1 (see, e.g., Table 1), and peptide library for HER2 ECD, PMA/ionomycin as a positive control, or DMSO as a mock stimulation (FIG. 6).

TABLE 1
ESR1 peptides

| ESR1 | Peptide Sequences | SEQ ID NO |
|---|---|---|
| ESR1 K303R | LWPSPLMIKRSKRNSLALSLTADQM | SEQ ID NO: 11 |
| ESR1 E380Q | VDLTLHDQVHLLQCAWLEILMIGLV | SEQ ID NO: 12 |
| ESR1 Y537N | SMKCKNVVPLNDLLLEMLDAHRL | SEQ ID NO: 13 |
| ESR1 Y537S | SMKCKNVVPLSDLLLEMLDAHRL | SEQ ID NO: 14 |
| ESR1 Y537C | SMKCKNVVPLCDLLLEMLDAHRL | SEQ ID NO: 15 |
| ESR1 D538G | SMKCKNVVPLYGLLLEMLDAHRL | SEQ ID NO: 16 |

To assess the in vivo impact of CHIKV- and SINV-derived vectors, in this experiment, they were compared to a VEE synthetic replicon derived from TC-83, which is commonly used within the field. In this example, several activating neoantigen mutations (encoded as ˜31-mer peptides centered around a mutation) were encoded for ESR1 and PI3K, a truncated tumor-associated antigen (extracellular and transmembrane domain of HER2, i.e. ERBB2 gene), and a kinase dead tumor-associated antigen (HER3, i.e. ERBB3 gene) on the same backbone. Similar to the Influenza study, the all LNP-formulated vectors demonstrated T cell responses in some or all of the mice against ESR1 mutations and HER2. Magnitude of total T cell responses as measured by ELISpot varies by each antigen. However, within responses to each individual antigen, in some cases individual vectors were significantly different from VEE, whereas in other cases each vector performs comparably to VEE (FIG. 6). This confirms that the new CHIKV- and SINV-vectors perform differently than VEE, and when combined with Example 4, demonstrates that the differences are unpredictable also dependent on the encoded protein. This confirms the utility of these vectors as advantaged for either vaccines or biotherapeutics on a target-by-target basis.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

1-54. (canceled)

55. A nucleic acid construct comprising a nucleic acid sequence encoding (i) a modified Chikungunya Virus (CHIKV) genome or replicon RNA or (ii) a modified Sindbis Virus (SINV) genome or replicon RNA, wherein the modified genome or replicon RNA is devoid of at least a portion of the nucleic acid sequence encoding one or more Viral structural proteins.

56. The nucleic acid construct of claim 55, further comprising one or more expression cassettes, wherein each of the expression cassettes comprises a promoter operably linked to a heterologous nucleic acid sequence.

57. The nucleic acid construct of claim 56, wherein at least one of the expression cassettes comprises a sub genomic (sg) promoter operably linked to the heterologous nucleic acid sequence.

58. The nucleic acid construct of claim 55, further comprising one or more untranslated regions (UTRs).

59. The nucleic acid construct of claim 58, wherein at least one of the UTRs is a heterologous UTR.

60. The nucleic acid construct of claim 55, wherein at least one of the expression cassettes comprises a coding sequence for a gene of interest (GOI).

61. The nucleic acid construct of claim 60, wherein the GOI encodes a polypeptide selected from the group consisting of a therapeutic polypeptide, a prophylactic polypeptide, a diagnostic polypeptide, a nutraceutical polypeptide, an industrial enzyme, and a reporter polypeptide.

62. The nucleic acid construct of claim 60, wherein the GOI encodes a polypeptide selected from the group consisting of an antibody, an antigen, an immune modulator, an enzyme, a signaling protein, and a cytokine.

63. The nucleic acid construct of claim 60, wherein the coding sequence of the GOI is optimized for expression at a level higher than the expression level of a reference coding sequence.

64. A recombinant cell comprising a nucleic acid construct according to claim 55.

65. A cell culture comprising at least one recombinant cell according to claim 64, and a culture medium.

66. A transgenic animal comprising a nucleic acid construct according to claim 55.

67. A method for producing a polypeptide of interest, comprising (i) culturing a recombinant cell comprising a nucleic acid construct according to claim 55, or (ii) rearing a transgenic animal comprising a recombinant cell of (i) under conditions wherein the transgenic animal or the recombinant cell produces the polypeptide encoded by the GOI.

68. A method for producing a polypeptide of interest in a subject, comprising administering to the subject a nucleic acid construct according to claim 55.

69. A recombinant polypeptide produced by the method of claim 68.

70. A pharmaceutical composition comprising a pharmaceutically acceptable excipient and:
(a) a nucleic acid construct of claim 55,
(b) a recombinant cell comprising the nucleic acid of (a), and/or
(c) a recombinant polypeptide encoded by the GOI of the nucleic acid of (a).

71. The pharmaceutical composition of claim 70, wherein the composition is formulated in a liposome, a lipid-based nanoparticle (LNP), or a polymer nanoparticle.

72. A method for eliciting an immune response, preventing and/or treating a health condition in a subject in need thereof, the method comprises administering to the subject a composition comprising:
(a) a nucleic acid construct of claim 55,
(b) a recombinant cell comprising the nucleic acid of (a),
(c) a recombinant polypeptide encoded by the nucleic acid of (a), and/or
(d) a pharmaceutical composition comprising one or more of the following: the nucleic acid of (a), the recombinant cell of (b), and the recombinant polypeptide of (c).

73. The method of claim 72, wherein the health condition is a proliferative disorder or a microbial infection.

74. A kit for eliciting an immune response, for the prevention, and/or for the treatment of a health condition or a microbial infection, the kit comprising:
(a) a nucleic acid construct of claim 55,
(b) a recombinant cell comprising the nucleic acid of (a),
(c) a recombinant polypeptide encoded by the G01 of the nucleic acid construct of (a), and/or
(d) a pharmaceutical composition comprising one or more of the following: the nucleic acid of (a), the recombinant cell of (b), and the recombinant polypeptide of (c).
